SEQUENCE LISTING (34 sequences)

SEQ ID NO: 1 - DNA, 72 bp, unidentified
  source 1..72: mol_type = unassigned DNA
gggggaggctgctggtgaatattaaccaaggtcaccccagttatcggaggagcaaacaggggctaagtccac

SEQ ID NO: 2 - DNA, 218 bp, unidentified
  source 1..218: mol_type = unassigned DNA
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccac

SEQ ID NO: 3 - DNA, 100 bp, unidentified
  source 1..100: mol_type = unassigned DNA
cactgggaggatgttgagtaagatggaaaactactgatgacccttgcagagacagagtattaggacatgtttgaacaggggccgggcgatcagcaggtag

SEQ ID NO: 4 - DNA, 200 bp, unidentified
  source 1..200: mol_type = unassigned DNA
ctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 5 - DNA, 82 bp, unidentified
  source 1..82: mol_type = unassigned DNA
gtaagggtttaagggatggttggttggtggggtattaatgtttaattacctggagcacctgcctgaaatcactttttttcag

SEQ ID NO: 6 - DNA, 131 bp, unidentified
  source 1..131: mol_type = unassigned DNA
acagaagtccactcattcttgaagaggtaagggtttaagggatggttggttggtggggtattaatgtttaattacctggagcacctgcctgaaatcactttttttcaggttggacgcgtgtcgacgccacc

SEQ ID NO: 7 - DNA, 672 bp, unidentified
  source 1..672: mol_type = unassigned DNA
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttgaagaggtaagggtttaagggatggttggttggtggggtattaatgtttaattacctggagcacctgcctgaaatcactttttttcaggttggacgcgtgtcgacgccacc

SEQ ID NO: 8 - DNA, 1035 bp, unidentified
  source 1..1035: mol_type = unassigned DNA
atggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatccattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggtaa

SEQ ID NO: 9 - protein, 344 aa, unidentified
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 10 - DNA, 541 bp, unidentified
  source 1..541: mol_type = unassigned DNA
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 11 - DNA, 395 bp, unidentified
  source 1..395: mol_type = unassigned DNA
gggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 12 - DNA, 324 bp, unidentified
  source 1..324: mol_type = unassigned DNA
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtag

SEQ ID NO: 13 - DNA, 317 bp, unidentified
  source 1..317: mol_type = unassigned DNA
cactgggaggatgttgagtaagatggaaaactactgatgacccttgcagagacagagtattaggacatgtttgaacaggggccgggcgatcagcaggtagctctagaggatccccgtctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 14 - DNA, 331 bp, unidentified
  source 1..331: mol_type = unassigned DNA
ctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttgaagaggtaagggtttaagggatggttggttggtggggtattaatgtttaattacctggagcacctgcctgaaatcactttttttcaggttggacgcgtgtcgacgccacc

SEQ ID NO: 15 - DNA, 21 bp, unidentified
  source 1..21: mol_type = unassigned DNA
acagaagtccactcattcttg

SEQ ID NO: 16 - skipped

SEQ ID NO: 17 - protein, 29 aa, unidentified
MVRARHQPGGLCLLLLLLCQFMEDRSAQA

SEQ ID NO: 18 - protein, 315 aa, unidentified
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 19 - protein, 317 aa, unidentified
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 20 - protein, 288 aa, unidentified
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 21 - protein, 344 aa, unidentified
MVRARHQPGGLCLLLLLLCQFMEDRSAQAGNCWLRQAKNGRCQVLYKTELSKEECYSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 22 - protein, 344 aa, unidentified
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKQQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 23 - protein, 74 aa, unidentified
GNCWLRQAKNGRCQVLYKTELSKEECCSTGRLSTSWTEEDVNDNTLFKWMIFNGGAPNCIPCKETCENVDCGPG

SEQ ID NO: 24 - protein, 24 aa, unidentified
TCENVDCGPGKKCRMNKKNKPRCV

SEQ ID NO: 25 - protein, 24 aa, unidentified
TCRDVFCPGSSTCVVDQTNNAYCV

SEQ ID NO: 26 - protein, 25 aa, unidentified
SCEDIQCTGGKKCLWDFKVGRGRCS

SEQ ID NO: 27 - protein, 55 aa, unidentified
NKPRCVCAPDCSNITWKGPVCGLDGKTYRNECALLKARCKEQPELEVQYQGRCKK

SEQ ID NO: 28 - protein, 56 aa, unidentified
NAYCVTCNRICPEPASSEQYLCGNDGVTYSSACHLRKATCLLGRSIGLAYEGKCIK

SEQ ID NO: 29 - protein, 55 aa, unidentified
RGRCSLCDELCPDSKSDEPVCASDNATYASECAMKEAACSSGVLLEVKHSGSCNS

SEQ ID NO: 30 - protein, 125 aa, unidentified
MGSGPSNFVNHAPGEAIVTGLEKGADKVAGTISHTIWEVIAGLVALLTFLAFGFWLFKYLQKRRERRRQLTEFQKRYLRNSYRLSEIQRPISQHEYEDPYEPPSRRKPPPPPYSTYVNIDNVSAI

SEQ ID NO: 31 - protein, 154 aa, unidentified
MGSGPSNFVNHAPGEAIVTGLEKGADKVAGTISHTIWEVIAGLVALLTFLAFGFWLFKYLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 32 - protein, 38 aa, unidentified
MGSGPSNFVNHAPGEAIVTGLEKGADKVAGTISHTIWE

SEQ ID NO: 33 - protein, 48 aa, unidentified
KLLQWYNRKSKNKKRKEQIREQIELGLLSYGAGVASLPLLNVIAHNPG

SEQ ID NO: 34 - protein, 48 aa, unidentified
VISATPIYKGPCTGVPNSRLLQITSGTAEENTRILNHDGRNPDGSINV

(19) United States
(12) Patent Application Publication
(10) Pub. No.: US 2024/0374760 A1
(43) Pub. Date: Nov. 14, 2024

(54) COMPOSITIONS AND METHODS FOR LIVER-SPECIFIC EXPRESSION OF FOLLISTATIN

(71) Applicant: OISIN BIOTECHNOLOGIES, INC., Seattle, WA (US)

(72) Inventors: Matthew Rein SCHOLZ, Seattle, WA (US); John David LEWIS, Encinitas, CA (US)

(21) Appl. No.: 18/620,618

(22) Filed: Mar. 28, 2024

Related U.S. Application Data
(63) Continuation of application No. PCT/US22/45468, filed on Sep. 30, 2022.
Application No. PCT/US22/45468 is a continuation-in-part of application No. PCT/CA2021/051377, filed on Oct. 1, 2021.

(51) Int. Cl. A61K 48/00; A61K 38/17; A61K 47/64; A61K 47/69; A61P 21/00

(52) U.S. Cl. CPC A61K 48/0058; A61K 38/1709; A61K 47/64; A61K 47/6907; A61K 48/0066; A61P 21/00

(57) ABSTRACT

Provided herein are expression constructs for liver-specific expression of transgenes, such as follistatin. Also provided are delivery vectors for the expression constructs, and methods of expressing follistatin in a subject to increase muscular strength and mass, or treat a condition.

### CROSS REFERENCE

This Application is a continuation of International Application No. PCT/US2022/45468, filed Sep. 30, 2022, which claims priority to and the benefit of International Application No. PCT/CA2021/051377, filed Oct. 1, 2021, each of which is incorporated herein by reference in its entirety.

### SEQUENCE LISTING STATEMENT

The instant application contains a Sequence Listing which has been submitted electronically in XML format and is hereby incorporated by reference in its entirety. Said XML copy, created on Mar. 28, 2024, is named 54636_714_301_SL.xml and is 36,412 bytes in size.

### SUMMARY

Disclosed herein, in some aspects, is an expression construct comprising an expression regulatory region operatively linked to a transgene that encodes follistatin, wherein the expression regulatory region comprises, from 5′ to 3′: (i) a CRM8 element and an engineered transthyretin enhancer (TTRe); or (ii) the TTRe and a human transthyretin promoter (hTTRm).

In some embodiments, the expression regulatory region comprises, from 5′ to 3′: the CRM8 element, the TTRe, and the hTTRm. In some embodiments, the hTTRm comprises a nucleotide sequence with at least 90% sequence identity to SEQ ID NO: 4.

Disclosed herein, in some aspects, is an expression construct comprising an expression regulatory region operatively linked to a transgene that encodes follistatin, wherein the expression regulatory region comprises a human transthyretin promoter (hTTRm) nucleotide sequence with at least 90% sequence identity to SEQ ID NO: 4.

In some embodiments, the expression regulatory region further comprises a CRM8 element. In some embodiments, the expression regulatory region further comprises an engineered transthyretin enhancer (TTRe). In some embodiments, the expression regulatory region further comprises an intron. In some embodiments, the intron is an MVM intron. In some embodiments, the CRM8 element comprises a nucleotide sequence with at least 90% sequence identity to SEQ ID NO: 1 or SEQ ID NO: 2. In some embodiments, the CRM8 element comprises a nucleotide sequence that is SEQ ID NO: 1 or SEQ ID NO: 2. In some embodiments, the TTRe comprises a nucleotide sequence with at least 90% sequence identity to SEQ ID NO: 3. In some embodiments, the TTRe comprises a nucleotide sequence that is SEQ ID NO: 3. In some embodiments, the hTTRm comprises a nucleotide sequence that is SEQ ID NO: 4. In some embodiments, the intron comprises a nucleotide sequence with at least 90% sequence identity to SEQ ID NO: 5. In some embodiments, the intron comprises a nucleotide sequence that is SEQ ID NO: 5. In some embodiments, the expression construct further comprises a hTTRm 5′ UTR. In some embodiments, the hTTRm 5′ UTR comprises the nucleotide sequence of SEQ ID NO: 15. In some embodiments, the expression regulatory region comprises a nucleotide sequence with at least 90% sequence identity to SEQ ID NO: 11. In some embodiments, the expression regulatory region comprises a nucleotide sequence with at least 95% sequence identity to SEQ ID NO: 11. In some embodiments, the expression regulatory region comprises a nucleotide sequence that is SEQ ID NO: 11. In some embodiments, the expression regulatory region comprises a nucleotide sequence with at least 90% sequence identity to SEQ ID NO: 7. In some embodiments, the expression regulatory region comprises a nucleotide sequence with at least 95% sequence identity to SEQ ID NO: 7. In some embodiments, the expression regulatory region comprises a nucleotide sequence that is SEQ ID NO: 7.

Disclosed herein, in some aspects, is an expression construct comprising an expression regulatory region that comprises a nucleotide sequence with at least 95% sequence identity to SEQ ID NO: 11.

In some embodiments, the expression regulatory region comprises a nucleotide sequence with at least 98% sequence identity to SEQ ID NO: 11. In some embodiments, the expression regulatory region comprises a nucleotide sequence that is SEQ ID NO: 11.

Disclosed herein, in some aspects, is an expression construct comprising an expression regulatory region that comprises a nucleotide sequence with at least 90% sequence identity to SEQ ID NO: 7.

In some embodiments, the expression regulatory region comprises a nucleotide sequence with at least 98% sequence identity to SEQ ID NO: 7. In some embodiments, the expression regulatory region comprises a nucleotide sequence that is SEQ ID NO: 7. In some embodiments, the expression regulatory region is operatively linked to a transgene. In some embodiments, the transgene encodes a therapeutic RNA or protein. In some embodiments, the transgene encodes follistatin. In some embodiments, the follistatin comprises an amino acid sequence with at least 90% sequence identity to any one of SEQ ID NOs: 23-29. In some embodiments, the follistatin comprises an amino acid sequence that is any one of SEQ ID NOs: 23-29. In some embodiments, the follistatin comprises the amino acid sequences of each of SEQ ID NOs: 23-29. In some embodiments, the follistatin comprises an amino acid sequence with at least 90% sequence identity to SEQ ID NO: 9 or SEQ ID NO: 18. In some embodiments, the follistatin comprises an amino acid sequence with at least 95% sequence identity to SEQ ID NO: 9 or SEQ ID NO: 18. In some embodiments, the follistatin comprises an amino acid sequence that is SEQ ID NO: 9 or SEQ ID NO: 18. In some embodiments, the follistatin comprises an amino acid sequence with at least 90% sequence identity to any one of SEQ ID NOs: 19-22. In some embodiments, the transgene comprises a nucleotide sequence with at least 90% sequence identity to SEQ ID NO: 8. In some embodiments, the transgene comprises a nucleotide sequence that is SEQ ID NO: 8. In some embodiments, the expression construct is a DNA plasmid, nanoplasmid, or minicircle.

Disclosed herein, in some aspects, is a vector comprising the expression construct of any one of the preceding embodiments.

In some embodiments, the vector is a non-viral vector. In some embodiments, the vector is a lipid-based vector. In some embodiments, the vector is a proteo-lipid vehicle (PLV), a lipid nanoparticle (LNP), or a liposome. In some embodiments, the vector comprises a fusogenic protein. In some embodiments, the vector is a PLV comprising an ionizable lipid and a fusion associated small transmembrane (FAST) protein.

Disclosed herein, in some aspects, is a cell comprising the expression construct of any one of the preceding embodiments.

Disclosed herein, in some aspects, is a pharmaceutical composition comprising the expression construct or vector of any one of the preceding embodiments, and a pharmaceutically-acceptable excipient, carrier, vehicle, or diluent.

Disclosed herein, in some aspects, is a method of treating a condition in a subject in need thereof, the method comprising administering to the subject an effective amount of the expression construct, the vector, the cell, or the pharmaceutical composition of any one of the preceding embodiments, thereby treating the condition.

Disclosed herein, in some aspects, is a method of increasing muscle mass or muscle strength in a subject, the method comprising administering to the subject an effective amount of the expression construct, the vector, the cell, or the pharmaceutical composition of any one of the preceding embodiments, thereby increasing the muscle mass or muscle strength in the subject.

Disclosed herein, in some aspects, is a method of promoting retention of muscle mass or muscle strength in a subject, the method comprising administering to the subject an effective amount of the expression construct, the vector, the cell, or the pharmaceutical composition of any one of the preceding embodiments, thereby the promoting retention of muscle mass or muscle strength in the subject.

Disclosed herein, in some aspects, is a method of reducing loss of muscle mass or muscle strength in a subject, the method comprising administering to the subject an effective amount of the expression construct, the vector, the cell, or the pharmaceutical composition of any one of the preceding embodiments, thereby reducing the loss of muscle mass or muscle strength in the subject.

Disclosed herein, in some aspects, is a method comprising administering an effective amount of the expression construct of any one of the preceding embodiments to a subject prior to a surgery, a period of immobilization, or a period of reduced mobility.

Disclosed herein, in some aspects, is a method of increasing muscle mass or strength in a subject, the method comprising administering to the subject an effective amount of an expression construct comprising an expression regulatory region operatively linked to a transgene that encodes follistatin, wherein the expression regulatory region induces preferential expression of the transgene in liver tissue, thereby increasing the muscle mass or muscle strength in the subject.

Disclosed herein, in some aspects, is a method of promoting retention of muscle mass or muscle strength in a subject, the method comprising administering to the subject an effective amount of an expression construct comprising an expression regulatory region operatively linked to a transgene that encodes follistatin, wherein the expression regulatory region induces preferential expression of the transgene in liver tissue, thereby promoting the retention of muscle mass or muscle strength in the subject.

Disclosed herein, in some aspects, is a method of reducing loss of muscle mass or muscle strength in a subject, the method comprising administering to the subject an effective amount of an expression construct comprising an expression regulatory region operatively linked to a transgene that encodes follistatin, wherein the expression regulatory region induces preferential expression of the transgene in liver tissue, thereby reducing the loss of muscle mass or muscle strength in the subject.

Disclosed herein, in some aspects, is a method comprising administering an effective amount of an expression construct to a subject prior to a surgery, a period of immobilization, or a period of reduced mobility, wherein the expression construct comprises an expression regulatory region operatively linked to a transgene that encodes follistatin, wherein the expression regulatory region induces preferential expression of the transgene in liver tissue

In some embodiments, the administering is parenteral. In some embodiments, the administering is systemic. In some embodiments, the administering is intravenous. In some embodiments, upon the administering to the subject, strength of a skeletal muscle of the subject is increased by at least 5% relative to before the administering. In some embodiments, upon the administering to the subject, strength of a skeletal muscle of the subject is increased by at least 5% relative to a control subject that is not administered the expression construct or is administered a control expression construct. In some embodiments, the strength of the skeletal muscle is as determined by a grip strength meter test. In some embodiments, upon the administering to the subject, mass of a skeletal muscle of the subject is increased by at least 5% relative to before the administering. In some embodiments, upon the administering to the subject, mass of a skeletal muscle of the subject is increased by at least 5% relative to a control subject that is not administered the expression construct or is administered a control expression construct. In some embodiments, the increase in muscle mass is as determined by imaging or Wheat germ agglutinin staining of muscle tissue. In some embodiments, the control expression construct comprises a CMV promoter that drives expression of follistatin. In some embodiments, the subject is a mammal. In some embodiments, the subject is a human. In some embodiments, the condition is or comprises age-related muscle loss, alpha-glucosidase deficiency, Becker muscular dystrophy, Duchenne muscular dystrophy (DMD), facioscapulohumeral muscular dystrophy, inclusion body myositis (IBM), inflammatory myopathy, muscular atrophy, muscular dystrophy, myopathy, myotonic dystrophy, sarcopenia, or sporadic inclusion body or myositis (sIBM). In some embodiments, the expression construct is encapsulated in a proteo-lipid vehicle (PLV) that comprises DOTAP:DODAP:DOPE:DMG-PEG at a mole percentage of 3:63:30:4. In some embodiments, the vector comprises the PLV, wherein the PLV comprises DOTAP:DODAP:DOPE:DMG-PEG at a mole percentage of 3:63:30:4.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A provides a circular map of a plasmid that comprises a promoter/enhancer sequence comprising, from 5′ to 3′: (i) three copies of a human CRM8 enhancer (part of the Serpinal promoter sequence, SEQ ID NO: 1), (ii) an engineered enhancer based on murine transthyretin enhancer elements (mTTRe, SEQ ID NO: 3), (iii) a minimal human transthyretin promoter (hTTRm, SEQ ID NO: 4), and (iv) an MVM intron (SEQ ID NO: 5). The promoter/enhancer also included a hTTRm 5′ UTR and a Kozak sequence, and some spacer nucleotides. The full sequence of the promoter/enhancer is provided in SEQ ID NO: 7. The promoter/enhancer was operatively linked to a nucleotide sequence encoding follistatin (FST/FST344, SEQ ID NO: 9, encoded by SEQ ID NO: 8).

FIG. 1B provides a linear map of the plasmid shown in FIG. 1A.

FIG. 2A illustrates levels of FST protein expression and phosphorylation of Akt and mTOR at 24, 48, and 72 hours after treatment or C2C12 mouse myoblasts with pDNA-CMV-FST (+) or pDNA-CMV-GFP (−). Numbers above the Western Blot phosphorylated bands represent fold increase relative to pDNA-CMV-GFP treated C2C12 lysates.

FIG. 2B shows media concentration of FST determined by ELISA after treatment of C2C12 mouse myoblasts with pDNA-CMV-FST PLVs.

FIG. 3 shows levels of FST detected in serum via ELISA after intravenous injection of PLVs containing expression constructs with FST expression driven by a transthyretin (TTR) promoter, a CMV promoter, an ApoE promoter, or a SerpEnh promoter.

FIG. 4A shows images of representative mice 15 weeks after intravenous injection of PBS (control) or PLVs containing 10 mg/kg expression constructs with FST expression driven by a TTR promoter or CMV promoter. Hair loss in PBS mouse is likely unrelated to treatment as all cage mates presented with the same condition.

FIG. 4B shows images of representative shaved mice after intravenous injection of PBS (control) or PLVs containing expression constructs with FST expression driven by a TTR promoter or CMV promoter.

FIG. 5A is a graph of body weight of mice 15 weeks following intravenous injection with PBS or FAST-PLVs encapsulating 10 mg/kg pDNA-CMV-FST or pDNA-TTR-FST. One-way ANOVA and Dunnett's multiple comparisons test, *P<0.05.

FIG. 5B is a graph showing percentage changes in body weight over time for mice after intravenous injection of PBS (control) or PLVs containing expression constructs with FST expression driven by a TTR promoter or CMV promoter. Body weight measurements from FIG. 5A were normalized to the initial body weight taken immediately prior to injection. One-way ANOVA and Dunnett's multiple comparisons test, *P<0.05.

FIG. 5C plots body weight over time for mice after intravenous injection of PBS (control) or PLVs containing expression constructs with FST expression driven by a TTR promoter or CMV promoter. Two-way ANOVA and Dunnett's multiple comparisons, relative to PBS group, *P<0.05, **P<0.01.

FIG. 5D plots percentage changes in body weight over time for mice after intravenous injection of PBS (control) or PLVs containing expression constructs with FST expression driven by a TTR promoter or CMV promoter. Two-way ANOVA and Dunnett's multiple comparisons test, **P<0.01.

FIG. 6A plots hindlimb grip strength of mice 15 weeks following intravenous injection with PBS or FAST-PLVs encapsulating 10 mg/kg pDNA-CMV-FST or pDNA-TTR-FST. One-way ANOVA and Dunnett's multiple comparisons test, **P<0.01.

FIG. 6B plots percentage changes in grip strength over time for mice after intravenous injection of PBS (control) or PLVs containing expression constructs with FST expression driven by a TTR promoter or CMV promoter. Grip strength measurements from FIG. 6A were normalized to the initial grip strength reading taken immediately prior to injection. One-way ANOVA and Dunnett's multiple comparisons test, **P<0.01.

FIG. 6C plots grip strength over time for mice after intravenous injection of PBS (control) or PLVs containing expression constructs with FST expression driven by a TTR promoter or CMV promoter. Two-way ANOVA and Dunnett's multiple comparisons, relative to PBS group, *P<0.05, **P<0.01, ***P<0.001, ****P<0.0001.

FIG. 6D plots percentage changes in grip strength over time for mice after intravenous injection of PBS (control) or PLVs containing expression constructs with FST expression driven by a TTR promoter or CMV promoter. Two-way ANOVA and Dunnett's multiple comparisons test, *P<0.05, **P<0.01, ***P<0.001, ****P<0.0001.

FIG. 7A provides gross dissection images of mice 34 weeks after intravenous injection of PBS (control) or PLVs containing expression constructs with FST expression driven by a TTR promoter or CMV promoter.

FIG. 7B plots the weight of gastrocnemius muscles of mice 34 weeks after intravenous injection of PBS (control) or PLVs containing expression constructs with FST expression driven by a TTR promoter or CMV promoter. One-way ANOVA and Dunnett's multiple comparisons test, **P<0.01.

FIG. 8A provides representative fluorescence microscopy images WGA-488 and DAPI-stained gastrocnemius muscle isolated from mice 34 weeks after intravenous injection of PBS (control) or PLVs containing expression constructs with FST expression driven by a TTR promoter or CMV promoter.

FIG. 8B plots the cross sectional muscle fiber area of gastrocnemius muscles of mice 34 weeks after intravenous injection of PBS (control) or PLVs containing expression constructs with FST expression driven by a TTR promoter or CMV promoter. Two-way ANOVA and Dunnett's multiple comparisons test, ****P<0.0001.

FIG. 9A shows hindlimb grip strength of mice 46 weeks after intramuscular injection of PBS (control) or PLVs containing 5 mg/kg an expression construct with FST expression driven by a CMV promoter. Unpaired t-test, *P<0.05.

FIG. 9B is a representative gross dissection image of a mouse 46 weeks after intramuscular injection of 5 mg/kg of an expression construct with FST expression driven by a CMV promoter. The injected side is indicated by an arrow.

FIG. 9C is a graph showing the weight of gastrocnemius muscles of mice 46 weeks after intramuscular injection of PBS (control) or PLVs containing expression constructs with FST expression driven by a CMV promoter. The ipsilateral side was injected. Unpaired t-test, **P<0.01.

FIG. 10A provides representative fluorescence microscopy images WGA-488 and DAPI-stained gastrocnemius muscle isolated from mice after intramuscular injection of PLVs containing expression constructs with FST expression driven by a CMV promoter. The ipsilateral side was injected.

FIG. 10B plots the cross sectional muscle fiber area of gastrocnemius muscles of mice after intramuscular injection of PLVs containing expression constructs with FST expression driven by a CMV promoter. The ipsilateral side was injected. Unpaired t-test, ****P<0.0001.

FIG. 11A plots hindlimb grip strength of mice 46 weeks after intravenous injection with 10 mg/kg pDNA-CMV-FST. Unpaired t-test, **P<0.01.

FIG. 11B provides images of gross dissection of mice 46 weeks after intravenous injection with PBS or 10 mg/kg pDNA-CMV-FST, with representative images of gastrocnemius WGA-488 staining.

FIG. 11C plots gastrocnemius weight 46 weeks after intravenous injection with pDNA-CMV-FST or PBS. Unpaired t-test, *P<0.05.

FIG. 11D plots cross-sectional muscle fiber area of WGA-488 stained gastrocnemius from mice injected intravenously with pDNA-CMV-FST or PBS, determined using MyoVision Software. Unpaired t-test, ****P<0.0001.

### DETAILED DESCRIPTION

There are a large number of clinical conditions and scenarios in which it would be beneficial to enhance muscular mass and strength, for example, to increase, retain, or reduce loss of muscular mass and strength.

Many genetic and aging-related conditions, including muscular dystrophies and sarcopenias, involve reductions in muscular mass and/or strength, which can result in weakness, decreased mobility, and a higher risk for injury. In addition, immobilization (e.g., associated with recovery from surgery or injury) can lead to muscular atrophy, which can prolong recovery and increase the risk of various complications. Therapeutic options to enhance muscular mass and strength are limited, and many are associated with adverse effects, limited efficacy, and a short therapeutic half-life.

Follistatin is a protein that can function as a potent inhibitor of the myostatin pathway, resulting in an increase in muscle mass and strength. Compositions and methods disclosed herein can be used to increase expression of follistatin, resulting in increases in muscle mass and strength, or reducing loss of muscle mass and strength. For example, expression constructs provided herein with a liver-specific promoter driving follistatin expression are shown to exhibit therapeutic effects with superior magnitude and duration compared to when a strong ubiquitous/constitutive promoter is used. In some cases, compositions and methods disclosed herein exhibit remarkably durable therapeutic effects, and require only a single or a limited number of doses.

### I. EXPRESSION CONSTRUCTS

The disclosure provides expression constructs that comprise an expression regulatory region, a transgene, or a combination thereof. An expression construct disclosed herein can be or can comprise DNA, for example, circular DNA or linear DNA. An expression construct disclosed herein can be or can comprise double stranded DNA. In some embodiments an expression construct is a plasmid. In some embodiments an expression construct is a nanoplasmid. In some embodiments an expression construct is a minicircle, a midge, a MIP, or a doggy bone. In some embodiments an expression construct comprises an R6K origin of replication. In some embodiments an expression construct lacks an origin of replication.

In some embodiments, an expression construct disclosed herein is not single stranded DNA. In some embodiments, an expression construct disclosed herein can be or can comprise single stranded DNA. In some embodiments, an expression construct disclosed herein lacks a component of a viral genome or lacks a viral packaging element, for example, lacks a 5′ and/or 3′ inverted terminal repeat (ITR). In some embodiments, an expression construct disclosed herein is non-integrating, e.g., does not integrate into the genome of a host cell.

An expression construct can be assembled by a variety of methods, e.g., by automated solid-phase synthesis. An expression construct can be constructed using standard solid-phase DNA/RNA synthesis An expression construct can also be constructed using a synthetic procedure. An expression construct can be synthesized manually or in a fully automated fashion. An expression construct can be a recombinant nucleic acid. In some cases, a synthetic procedure can comprise 5′-hydroxyl oligonucleotides that can be initially transformed into corresponding 5′-H-phosphonate mono esters, subsequently oxidized in the presence of imidazole to activated 5′-phosphorimidazolidates, and finally reacted with pyrophosphate on a solid support. This procedure can include a purification step after the synthesis such as PAGE, HPLC, MS, or any combination thereof. Polynucleotides can be purchased commercially.

In some embodiments, an expression construct disclosed herein comprises natural, synthetic, and/or artificial nucleotide analogues or bases. In some embodiments, the synthetic or artificial nucleotide analogues or bases comprise modifications at one or more of a deoxyribose moiety, ribose moiety, phosphate moiety, nucleoside moiety, or a combination thereof. In some embodiments, a nucleotide analogue comprises a modified base. In some embodiments, one or more modifications optionally occur at the internucleotide linkage. In some embodiments, one or more modifications comprise a modified phosphate backbone in which the modification generates a neutral or uncharged backbone.

### A. Expression Regulatory Region

An expression construct disclosed herein can comprise an expression regulatory region. An expression regulatory region can comprise, for example, a cis-regulatory element (e.g., a CRM8 element derived from the SERPINA1 gene or promoter), an enhancer (e.g., an engineered transthyretin enhancer (TTRe)), a promoter (e.g., a human transthyretin promoter (hTTRm)), and/or an intron (e.g., a minute virus of mouse (MVM) intron). The expression regulatory region can be operatively linked to a transgene, such that the expression regulatory region regulates expression (e.g., transcription and/or translation) of the transgene.

In some embodiments, an expression regulatory region comprises, consists essentially of, or consists of, (i) a cis-regulatory element, (ii) an enhancer, (iii) a promoter, and (iv) an intron.

In some embodiments, an expression regulatory region comprises, consists essentially of, or consists of, (i) an enhancer, (ii) a promoter, and (iii) an intron. In some embodiments, an expression regulatory region comprises, consists essentially of, or consists of, (i) a cis-regulatory element, (ii) a promoter, and (iii) an intron. In some embodiments, an expression regulatory region comprises, consists essentially of, or consists of, (i) a cis-regulatory element, (ii) an enhancer, and (iii) an intron. In some embodiments, an expression regulatory region comprises, consists essentially of, or consists of, (i) a cis-regulatory element, (ii) an enhancer, and (iii) a promoter.

In some embodiments, an expression regulatory region comprises, consists essentially of, or consists of, (i) a promoter, and (ii) an intron. In some embodiments, an expression regulatory region comprises, consists essentially of, or consists of, (i) a cis-regulatory element, and (ii) an intron. In some embodiments, an expression regulatory region comprises, consists essentially of, or consists of, (i) an enhancer, and (ii) an intron. In some embodiments, an expression regulatory region comprises, consists essentially of, or consists of, (i) a cis-regulatory element, and (ii) a promoter. In some embodiments, an expression regulatory region comprises, consists essentially of, or consists of, (i) an enhancer, and (ii) a promoter. In some embodiments, an expression regulatory region comprises, consists essentially of, or consists of, (i) a cis-regulatory element, and (ii) an enhancer.

In some embodiments, an expression regulatory region comprises, consists essentially of, or consists of, from 5′ to 3′, (i) a cis-regulatory element, (ii) an enhancer, (iii) a promoter, and (iv) an intron.

In some embodiments, an expression regulatory region comprises, consists essentially of, or consists of, from 5′ to 3′, (i) an enhancer, (ii) a promoter, and (iii) an intron. In some embodiments, an expression regulatory region comprises, consists essentially of, or consists of, from 5′ to 3′, (i) a cis-regulatory element, (ii) a promoter, and (iii) an intron. In some embodiments, an expression regulatory region comprises, consists essentially of, or consists of, from 5′ to 3′, (i) a cis-regulatory element, (ii) an enhancer, and (iii) an intron. In some embodiments, an expression regulatory region comprises, consists essentially of, or consists of, from 5′ to 3′, (i) a cis-regulatory element, (ii) an enhancer, and (iii) a promoter.

In some embodiments, an expression regulatory region comprises, consists essentially of, or consists of, from 5′ to 3′, (i) a promoter, and (ii) an intron. In some embodiments, an expression regulatory region comprises, consists essentially of, or consists of, from 5′ to 3′, (i) a cis-regulatory element, and (ii) an intron. In some embodiments, an expression regulatory region comprises, consists essentially of, or consists of, from 5′ to 3′, (i) an enhancer, and (ii) an intron.

In some embodiments, an expression regulatory region comprises, consists essentially of, or consists of, from 5′ to 3′, (i) a cis-regulatory element, and (ii) a promoter. In some embodiments, an expression regulatory region comprises, consists essentially of, or consists of, from 5′ to 3′, (i) an enhancer, and (ii) a promoter.

In some embodiments, an expression regulatory region comprises, consists essentially of, or consists of, from 5′ to 3′, (i) a cis-regulatory element, and (ii) an enhancer.

In some embodiments, an expression regulatory region comprises, consists essentially of, or consists of, (i) CRM8, (ii) TTRe, (iii) hTTRm, and (iv) MVM intron.

In some embodiments, an expression regulatory region comprises, consists essentially of, or consists of, (i) TTRe, (ii) hTTRm, and (iii) MVM intron. In some embodiments, an expression regulatory region comprises, consists essentially of, or consists of, (i) CRM8, (ii) hTTRm, and (iii) MVM intron. In some embodiments, an expression regulatory region comprises, consists essentially of, or consists of, (i) CRM8, (ii) TTRe, and (iii) MVM intron. In some embodiments, an expression regulatory region comprises, consists essentially of, or consists of, (i) CRM8, (ii) TTRe, and (iii) hTTRm.

In some embodiments, an expression regulatory region comprises, consists essentially of, or consists of, (i) hTTRm, and (ii) MVM intron. In some embodiments, an expression regulatory region comprises, consists essentially of, or consists of, (i) CRM8, and (ii) MVM intron. In some embodiments, an expression regulatory region comprises, consists essentially of, or consists of, (i) TTRe, and (ii) MVM intron. In some embodiments, an expression regulatory region comprises, consists essentially of, or consists of, (i) CRM8, and (ii) hTTRm. In some embodiments, an expression regulatory region comprises, consists essentially of, or consists of, (i) TTRe, and (ii) hTTRm. In some embodiments, an expression regulatory region comprises, consists essentially of, or consists of, (i) CRM8, and (ii) TTRe.

In some embodiments, an expression regulatory region comprises, consists essentially of, or consists of, from 5′ to 3′, (i) CRM8, (ii) TTRe, (iii) hTTRm, and (iv) MVM intron.

In some embodiments, an expression regulatory region comprises, consists essentially of, or consists of, from 5′ to 3′, (i) TTRe, (ii) hTTRm, and (iii) MVM intron. In some embodiments, an expression regulatory region comprises, consists essentially of, or consists of, from 5′ to 3′, (i) CRM8, (ii) hTTRm, and (iii) MVM intron. In some embodiments, an expression regulatory region comprises, consists essentially of, or consists of, from 5′ to 3′, (i) CRM8, (ii) TTRe, and (iii) MVM intron. In some embodiments, an expression regulatory region comprises, consists essentially of, or consists of, from 5′ to 3′, (i) CRM8, (ii) TTRe, and (iii) hTTRm.

In some embodiments, an expression regulatory region comprises, consists essentially of, or consists of, from 5′ to 3′, (i) hTTRm, and (ii) MVM intron. In some embodiments, an expression regulatory region comprises, consists essentially of, or consists of, from 5′ to 3′, (i) CRM8, and (ii) MVM intron. In some embodiments, an expression regulatory region comprises, consists essentially of, or consists of, from 5′ to 3′, (i) TTRe, and (ii) MVM intron.

In some embodiments, an expression regulatory region comprises, consists essentially of, or consists of, from 5′ to 3′, (i) CRM8, and (ii) hTTRm. In some embodiments, an expression regulatory region comprises, consists essentially of, or consists of, from 5′ to 3′, (i) TTRe, and (ii) hTTRm.

In some embodiments, an expression regulatory region comprises, consists essentially of, or consists of, from 5′ to 3′, (i) CRM8, and (ii) TTRe.

An expression construct disclosed herein can comprise multiple expression regulatory regions, for example two expression regulatory regions, or more.

### 1. Cis-Regulatory Element

An expression regulatory region disclosed herein can comprise a cis-regulatory element, for example, a CRM8 element derived from the SERPINA1 gene or promoter. A cis-regulatory element can be a liver-specific (e.g., hepatocyte-specific) cis-regulatory module.

A cis-regulatory element can be an engineered cis-regulatory element. A cis-regulatory element can be a synthetic cis-regulatory element. A cis-regulatory element can be a cis-regulatory element as found in a naturally-occurring genome. In some embodiments, a cis-regulatory element is not found in a naturally-occurring genome. A cis-regulatory element can be a mammalian cis-regulatory element or derived from a mammalian cis-regulatory element. A cis-regulatory element can be a human cis-regulatory element or derived from a human cis-regulatory element. A cis-regulatory element can be a murine cis-regulatory element or derived from a murine cis-regulatory element. A cis-regulatory element can be a liver-specific (e.g., hepatocyte-specific) cis-regulatory element.

A cis-regulatory element (e.g., CRM8 element) disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to SEQ ID NO: 1 or SEQ ID NO: 2.

In some embodiments, a cis-regulatory element (e.g., CRM8 element) comprises, consists essentially of, or consists of a nucleotide sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity to SEQ ID NO: 1 or SEQ ID NO: 2.

In some embodiments, a cis-regulatory element (e.g., CRM8 element) comprises, consists essentially of, or consists of a nucleotide sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity to SEQ ID NO: 1 or SEQ ID NO: 2.

In some embodiments, a cis-regulatory element (e.g., CRM8 element) comprises the nucleotide sequence of SEQ ID NO: 1. In some embodiments, a cis-regulatory element (e.g., CRM8 element) comprises a nucleotide sequence that consists essentially of SEQ ID NO: 1. In some embodiments, a cis-regulatory element (e.g., CRM8 element) comprises a nucleotide sequence that consists of SEQ ID NO: 1. In some embodiments, a cis-regulatory element (e.g., CRM8 element) consists essentially of SEQ ID NO: 1. In some embodiments, a cis-regulatory element (e.g., CRM8 element) consists of SEQ ID NO: 1.

In some embodiments, a cis-regulatory element (e.g., CRM8 element) comprises, consists essentially of, or consists of two or more copies of SEQ ID NO: 1 or a nucleotide sequence with a degree of sequence identity disclosed herein, for example, 2, 3, 4, or 5 copies. The copies can be separated by a single spacing nucleotide. The copies can be separated by two spacing nucleotides. The copies can be separated by 3, 4, 5, 6, 7, 8, 9, or 10 spacing nucleotides. In some embodiments, no spacing nucleotide is present between the copies.

In some embodiments, a cis-regulatory element (e.g., CRM8 element) comprises the nucleotide sequence of SEQ ID NO: 2. In some embodiments, a cis-regulatory element (e.g., CRM8 element) comprises a nucleotide sequence that consists essentially of SEQ ID NO: 2. In some embodiments, a cis-regulatory element (e.g., CRM8 element) comprises a nucleotide sequence that consists of SEQ ID NO: 2. In some embodiments, a cis-regulatory element (e.g., CRM8 element) consists essentially of SEQ ID NO: 2. In some embodiments, a cis-regulatory element (e.g., CRM8 element) consists of SEQ ID NO: 2.

In some embodiments, a cis-regulatory element (e.g., CRM8 element) comprises a nucleotide sequence with one or more insertions, deletions, and/or substitutions relative to SEQ ID NO: 1 or SEQ ID NO: 2. For example, a cis-regulatory element (e.g., CRM8 element) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions relative to SEQ ID NO: 1 or SEQ ID NO: 2.

In some embodiments, a cis-regulatory element (e.g., CRM8 element) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to SEQ ID NO: 1 or SEQ ID NO: 2.

In some embodiments, a cis-regulatory element (e.g., CRM8 element) comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to SEQ ID NO: 1 or SEQ ID NO: 2.

The one or more insertions can be at the 5′ end, the 3′ end, within the nucleotide sequence, or a combination thereof. The one or more insertions can be contiguous, non-contiguous, or a combination thereof.

A cis-regulatory element (e.g., CRM8 element) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide deletions relative to SEQ ID NO: 1 or SEQ ID NO: 2.

In some embodiments, a cis-regulatory element (e.g., CRM8 element) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide deletions relative to SEQ ID NO: 1 or SEQ ID NO: 2.

In some embodiments, a cis-regulatory element (e.g., CRM8 element) comprises 1, 2, 3, 4, 5, 6, 7, 8, 9, 10, 11, 12, 13, 14, 15, 16, 17, 18, 19, 20, 25, 30, 35, 40, 45, or 50 nucleotide deletions relative to SEQ ID NO: 1 or SEQ ID NO: 2.

The one or more deletions can be at the 5′ end, the 3′ end, within the nucleotide sequence, or a combination thereof. The one or more deletions can be contiguous, non-contiguous, or a combination thereof.

A cis-regulatory element (e.g., CRM8 element) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide substitutions relative to SEQ ID NO: 1 or SEQ ID NO: 2.

In some embodiments, a cis-regulatory element (e.g., CRM8 element) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to SEQ ID NO: 1 or SEQ ID NO: 2.

In some embodiments, a cis-regulatory element (e.g., CRM8 element) comprises 1, 2, 3, 4, 5, 6, 7, 8, 9, 10, 11, 12, 13, 14, 15, 16, 17, 18, 19, 20, 25, 30, 35, 40, 45, or 50 nucleotide substitutions relative to SEQ ID NO: 1 or SEQ ID NO: 2.

The one or more substitutions can be at the 5′ end, the 3′ end, within the nucleotide sequence, or a combination thereof. The one or more substitutions can be contiguous, non-contiguous, or a combination thereof.

An expression regulatory region disclosed herein can comprise any suitable number of cis-regulatory elements. An expression regulatory region can comprise at least 1, at least 2, at least 3, at least 4, or at least 5 cis-regulatory elements. An expression regulatory region can contain at most 1, at most 2, at most 3, at most 4, or at most 5 cis-regulatory elements. An expression regulatory region can comprise 1, 2, 3, 4, or 5 cis-regulatory elements.

An expression regulatory region disclosed herein can comprise any suitable number of CRM8 elements. An expression regulatory region can comprise at least 1, at least 2, at least 3, at least 4, or at least 5 CRM8 elements. An expression regulatory region can contain at most 1, at most 2, at most 3, at most 4, or at most 5 CRM8 elements. An expression regulatory region can comprise 1, 2, 3, 4, or 5 CRM8 elements.

### 2. Enhancer

An expression regulatory region disclosed herein can comprise an enhancer, for example, an engineered transthyretin enhancer (TTRe).

An enhancer can be an engineered enhancer. An enhancer can be a synthetic enhancer. An enhancer can be an enhancer as found in a naturally-occurring genome. In some embodiments, an enhancer is not found in a naturally-occurring genome. An enhancer can be a mammalian enhancer or derived from a mammalian enhancer. An enhancer can be a human enhancer or derived from a human enhancer. An enhancer can be a murine enhancer or derived from a murine enhancer. An enhancer can be a liver-specific (e.g., hepatocyte-specific) enhancer. An enhancer can be a ubiquitous or constitutive enhancer.

An enhancer can be, for example, an engineered transthyretin enhancer (TTRe).

An enhancer (e.g., TTRe) disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to SEQ ID NO: 3.

In some embodiments, an enhancer (e.g., TTRe) comprises, consists essentially of, or consists of a nucleotide sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity to SEQ ID NO: 3.

In some embodiments, an enhancer (e.g., TTRe) comprises, consists essentially of, or consists of a nucleotide sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity to SEQ ID NO: 3.

In some embodiments, an enhancer (e.g., TTRe) comprises, consists essentially of, or consists of the nucleotide sequence of SEQ ID NO: 3. In some embodiments, an enhancer (e.g., TTRe) comprises a nucleotide sequence that consists essentially of SEQ ID NO: 3. In some embodiments, an enhancer (e.g., TTRe) comprises a nucleotide sequence that consists of SEQ ID NO: 3. In some embodiments, an enhancer (e.g., TTRe) consists essentially of SEQ ID NO: 3. In some embodiments, an enhancer (e.g., TTRe) consists of SEQ ID NO: 3.

In some embodiments, an enhancer (e.g., TTRe) comprises a nucleotide sequence with one or more insertions, deletions, and/or substitutions relative to SEQ ID NO: 3. For example, an enhancer (e.g., TTRe) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions relative to SEQ ID NO: 3.

In some embodiments, an enhancer (e.g., TTRe) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to SEQ ID NO: 3.

In some embodiments, an enhancer (e.g., TTRe) comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to SEQ ID NO: 3.

The one or more insertions can be at the 5′ end, the 3′ end, within the nucleotide sequence, or a combination thereof. The one or more insertions can be contiguous, non-contiguous, or a combination thereof.

An enhancer (e.g., TTRe) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide deletions relative to SEQ ID NO: 3.

In some embodiments, an enhancer (e.g., TTRe) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide deletions relative to SEQ ID NO: 3.

In some embodiments, an enhancer (e.g., TTRe) comprises 1, 2, 3, 4, 5, 6, 7, 8, 9, 10, 11, 12, 13, 14, 15, 16, 17, 18, 19, 20, 25, 30, 35, 40, 45, or 50 nucleotide deletions relative to SEQ ID NO: 3.

The one or more deletions can be at the 5′ end, the 3′ end, within the nucleotide sequence, or a combination thereof. The one or more deletions can be contiguous, non-contiguous, or a combination thereof.

An enhancer (e.g., TTRe) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide substitutions relative to SEQ ID NO: 3.

In some embodiments, an enhancer (e.g., TTRe) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to SEQ ID NO: 3.

In some embodiments, an enhancer (e.g., TTRe) comprises 1, 2, 3, 4, 5, 6, 7, 8, 9, 10, 11, 12, 13, 14, 15, 16, 17, 18, 19, 20, 25, 30, 35, 40, 45, or 50 nucleotide substitutions relative to SEQ ID NO: 3.

The one or more substitutions can be at the 5′ end, the 3′ end, within the nucleotide sequence, or a combination thereof. The one or more substitutions can be contiguous, non-contiguous, or a combination thereof.

An expression regulatory region disclosed herein can comprise any suitable number of TTRe elements. An expression regulatory region can comprise at least 1, at least 2, at least 3, at least 4, or at least 5 TTRe elements. An expression regulatory region can contain at most 1, at most 2, at most 3, at most 4, or at most 5 TTRe elements. An expression regulatory region can comprise 1, 2, 3, 4, or 5 TTRe elements.

An expression regulatory region disclosed herein can comprise any suitable number of enhancers. An expression regulatory region can comprise at least 1, at least 2, at least 3, at least 4, or at least 5 enhancers. An expression regulatory region can contain at most 1, at most 2, at most 3, at most 4, or at most 5 enhancers. An expression regulatory region can comprise 1, 2, 3, 4, or 5 enhancers.

### 3. Promoter

An expression regulatory region disclosed herein can comprise a promoter, for example, a human transthyretin promoter (hTTRm). The promoter can be an engineered promoter. The promoter can be a synthetic promoter. The promoter can be a promoter as found in a naturally-occurring genome. In some embodiments, a promoter is not found in a naturally-occurring genome. The promoter can be a mammalian promoter or derived from a mammalian promoter. The promoter can be a human promoter or derived from a human promoter. The promoter can be a murine promoter or derived from a murine promoter. The promoter can be a constitutive promoter. The promoter can be a ubiquitous promoter. The promoter can be an inducible promoter. The promoter can be a tissue-specific or cell type-specific promoter. The promoter can be a liver-specific (e.g., hepatocyte-specific) promoter. The promoter can be a minimal promoter.

A promoter (e.g., hTTRm) disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to SEQ ID NO: 4.

In some embodiments, a promoter (e.g., hTTRm) comprises, consists essentially of, or consists of a nucleotide sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5% at most about 98%, at most about 98.5%, at most about 99%, or at most about 99.5% sequence identity to SEQ ID NO: 4.

In some embodiments, a promoter (e.g., hTTRm) comprises, consists essentially of, or consists of a nucleotide sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity to SEQ ID NO: 4.

In some embodiments, a promoter (e.g., hTTRm) comprises the nucleotide sequence of SEQ ID NO: 4. In some embodiments, a promoter (e.g., hTTRm) comprises a nucleotide sequence that consists essentially of SEQ ID NO: 4. In some embodiments, a promoter (e.g., hTTRm) comprises a nucleotide sequence that consists of SEQ ID NO: 4. In some embodiments, a promoter (e.g., hTTRm) consists essentially of SEQ ID NO: 4. In some embodiments, a promoter (e.g., hTTRm) consists of SEQ ID NO: 4.

In some embodiments, a promoter (e.g., hTTRm) comprises a nucleotide sequence with one or more insertions, deletions, and/or substitutions relative to SEQ ID NO: 4. For example, a promoter (e.g., hTTRm) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions relative to SEQ ID NO: 4.

In some embodiments, a promoter (e.g., hTTRm) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to SEQ ID NO: 4.

In some embodiments, a promoter (e.g., hTTRm) comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to SEQ ID NO: 4.

The one or more insertions can be at the 5′ end, the 3′ end, within the nucleotide sequence, or a combination thereof. The one or more insertions can be contiguous, non-contiguous, or a combination thereof.

A promoter (e.g., hTTRm) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide deletions relative to SEQ ID NO: 4.

In some embodiments, a promoter (e.g., hTTRm) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide deletions relative to SEQ ID NO: 4.

In some embodiments, a promoter (e.g., hTTRm) comprises 1, 2, 3, 4, 5, 6, 7, 8, 9, 10, 11, 12, 13, 14, 15, 16, 17, 18, 19, 20, 25, 30, 35, 40, 45, or 50 nucleotide deletions relative to SEQ ID NO: 4.

The one or more deletions can be at the 5′ end, the 3′ end, within the nucleotide sequence, or a combination thereof. The one or more deletions can be contiguous, non-contiguous, or a combination thereof.

A promoter (e.g., hTTRm) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide substitutions relative to SEQ ID NO: 4.

In some embodiments, a promoter (e.g., hTTRm) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to SEQ ID NO: 4.

In some embodiments, a promoter (e.g., hTTRm) comprises 1, 2, 3, 4, 5, 6, 7, 8, 9, 10, 11, 12, 13, 14, 15, 16, 17, 18, 19, 20, 25, 30, 35, 40, 45, or 50 nucleotide substitutions relative to SEQ ID NO: 4.

The one or more substitutions can be at the 5′ end, the 3′ end, within the nucleotide sequence, or a combination thereof. The one or more substitutions can be contiguous, non-contiguous, or a combination thereof.

An expression regulatory region disclosed herein can comprise any suitable number of promoters. An expression regulatory region can comprise at least 1, at least 2, at least 3, at least 4, or at least 5 promoters. An expression regulatory region can contain at most 1, at most 2, at most 3, at most 4, or at most 5 promoters. An expression regulatory region can comprise 1, 2, 3, 4, or 5 promoters.

An expression regulatory region disclosed herein can comprise any suitable number of hTTRm elements. An expression regulatory region can comprise at least 1, at least 2, at least 3, at least 4, or at least 5 hTTRm elements. An expression regulatory region can contain at most 1, at most 2, at most 3, at most 4, or at most 5 hTTRm elements. An expression regulatory region can comprise 1, 2, 3, 4, or 5 hTTRm elements.

### 4. Intron

An expression regulatory region disclosed herein can comprise an intron, for example, an MVM intron. The intron can be an engineered intron. The intron can be a synthetic intron. The intron can be an intron as found in a naturally-occurring genome. In some embodiments, an intron is not found in a naturally-occurring genome. The intron can be a mammalian intron or derived from a mammalian intron. The intron can be a human intron or derived from a human intron. The intron can be a murine intron or derived from a murine intron. An intron can be truncated relative to a wild type intron. An intron can be chimeric, for example, comprising a portion of a first intron and a portion of a second intron.

An intron can be or can comprise, for example, a minute virus of mouse (MVM), pCI, or beta-globin intron.

An intron (e.g., MVM) disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to SEQ ID NO: 5 or SEQ ID NO: 6.

In some embodiments, an intron (e.g., MVM) comprises, consists essentially of, or consists of a nucleotide sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5% at most about 98%, at most about 98.5%, at most about 99%, or at most about 99.5% sequence identity to SEQ ID NO: 5 or SEQ ID NO: 6.

In some embodiments, an intron (e.g., MVM) comprises, consists essentially of, or consists of a nucleotide sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity to SEQ ID NO: 5 or SEQ ID NO: 6.

In some embodiments, an intron (e.g., MVM) comprises the nucleotide sequence of SEQ ID NO: 5. In some embodiments, an intron (e.g., MVM) comprises a nucleotide sequence that consists essentially of SEQ ID NO: 5. In some embodiments, an intron (e.g., MVM) comprises a nucleotide sequence that consists of SEQ ID NO: 5. In some embodiments, an intron (e.g., MVM) consists essentially of SEQ ID NO: 5. In some embodiments, an intron (e.g., MVM) consists of SEQ ID NO: 5.

In some embodiments, an intron (e.g., MVM) comprises the nucleotide sequence of SEQ ID NO: 6. In some embodiments, an intron (e.g., MVM) comprises a nucleotide sequence that consists essentially of SEQ ID NO: 6. In some embodiments, an intron (e.g., MVM) comprises a nucleotide sequence that consists of SEQ ID NO: 6. In some embodiments, an intron (e.g., MVM) consists essentially of SEQ ID NO: 6. In some embodiments, an intron (e.g., MVM) consists of SEQ ID NO: 6.

In some embodiments, an intron (e.g., MVM) comprises a nucleotide sequence with one or more insertions, deletions, and/or substitutions relative to SEQ ID NO: 5 or SEQ ID NO: 6. For example, an intron (e.g., MVM) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions relative to SEQ ID NO: 5 or SEQ ID NO: 6.

In some embodiments, an intron (e.g., MVM) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to SEQ ID NO: 5 or SEQ ID NO: 6.

In some embodiments, an intron (e.g., MVM) comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to SEQ ID NO: 5 or SEQ ID NO: 6.

The one or more insertions can be at the 5′ end, the 3′ end, within the nucleotide sequence, or a combination thereof. The one or more insertions can be contiguous, non-contiguous, or a combination thereof.

An intron (e.g., MVM) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide deletions relative to SEQ ID NO: 5 or SEQ ID NO: 6.

In some embodiments, an intron (e.g., MVM) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide deletions relative to SEQ ID NO: 5 or SEQ ID NO: 6.

In some embodiments, an intron (e.g., MVM) comprises 1, 2, 3, 4, 5, 6, 7, 8, 9, 10, 11, 12, 13, 14, 15, 16, 17, 18, 19, 20, 25, 30, 35, 40, 45, or 50 nucleotide deletions relative to SEQ ID NO: 5 or SEQ ID NO: 6.

The one or more deletions can be at the 5′ end, the 3′ end, within the nucleotide sequence, or a combination thereof. The one or more deletions can be contiguous, non-contiguous, or a combination thereof.

An intron (e.g., MVM) can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide substitutions relative to SEQ ID NO: 5 or SEQ ID NO: 6.

In some embodiments, an intron (e.g., MVM) comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to SEQ ID NO: 5 or SEQ ID NO: 6.

In some embodiments, an intron (e.g., MVM) comprises 1, 2, 3, 4, 5, 6, 7, 8, 9, 10, 11, 12, 13, 14, 15, 16, 17, 18, 19, 20, 25, 30, 35, 40, 45, or 50 nucleotide substitutions relative to SEQ ID NO: 5 or SEQ ID NO: 6.

The one or more substitutions can be at the 5′ end, the 3′ end, within the nucleotide sequence, or a combination thereof. The one or more substitutions can be contiguous, non-contiguous, or a combination thereof.

An expression regulatory region disclosed herein can comprise any suitable number of MVM introns. An expression regulatory region can comprise at least 1, at least 2, at least 3, at least 4, or at least 5 MVM introns. An expression regulatory region can contain at most 1, at most 2, at most 3, at most 4, or at most 5 MVM introns. An expression regulatory region can comprise 1, 2, 3, 4, or 5 MVM introns.

An expression regulatory region disclosed herein can comprise any suitable number of introns. An expression regulatory region can comprise at least 1, at least 2, at least 3, at least 4, or at least 5 introns. An expression regulatory region can contain at most 1, at most 2, at most 3, at most 4, or at most 5 introns. An expression regulatory region can comprise 1, 2, 3, 4, or 5 introns.

### 5. Additional Sequences

An expression regulatory region disclosed herein can comprise, consist essentially of, or consist of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 7 and 10-16.

In some embodiments, an expression regulatory region comprises, consists essentially of, or consists of a nucleotide sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity to any one of SEQ ID NOs: 7 and 10-16.

In some embodiments, an expression regulatory region comprises, consists essentially of, or consists of a nucleotide sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity to any one of SEQ ID NOs: 7 and 10-16.

In some embodiments, an expression regulatory region comprises the nucleotide sequence of any one of SEQ ID NOs: 7 and 10-16. In some embodiments, an expression regulatory region comprises a nucleotide sequence that consists essentially of any one of SEQ ID NOs: 7 and 10-16. In some embodiments, an expression regulatory region comprises a nucleotide sequence that consists of any one of SEQ ID NOs: 7 and 10-16. In some embodiments, an expression regulatory region consists essentially of any one of SEQ ID NOs: 7 and 10-16. In some embodiments, an expression regulatory region consists of any one of SEQ ID NOs: 7 and 10-16.

In some embodiments, an expression regulatory region comprises a nucleotide sequence with one or more insertions, deletions, and/or substitutions relative to any one of SEQ ID NOs: 7 and 10-16. For example, an expression regulatory region can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide insertions relative to any one of SEQ ID NOs: 7 and 10-16.

In some embodiments, an expression regulatory region comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to any one of SEQ ID NOs: 7 and 10-16.

In some embodiments, an expression regulatory region comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to any one of SEQ ID NOs: 7 and 10-16.

The one or more insertions can be at the 5′ end, the 3′ end, within the nucleotide sequence, or a combination thereof. The one or more insertions can be contiguous, non-contiguous, or a combination thereof.

An expression regulatory region can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide deletions relative to any one of SEQ ID NOs: 7 and 10-16.

In some embodiments, an expression regulatory region comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide deletions relative to any one of SEQ ID NOs: 7 and 10-16.

In some embodiments, an expression regulatory region comprises 1, 2, 3, 4, 5, 6, 7, 8, 9, 10, 11, 12, 13, 14, 15, 16, 17, 18, 19, 20, 25, 30, 35, 40, 45, or 50 nucleotide deletions relative to any one of SEQ ID NOs: 7 and 10-16.

The one or more deletions can be at the 5′ end, the 3′ end, within the nucleotide sequence, or a combination thereof. The one or more deletions can be contiguous, non-contiguous, or a combination thereof.

An expression regulatory region can comprise a nucleotide sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 nucleotide substitutions relative to any one of SEQ ID NOs: 7 and 10-16.

In some embodiments, an expression regulatory region comprises a nucleotide sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to any one of SEQ ID NOs: 7 and 10-16.

In some embodiments, an expression regulatory region comprises 1, 2, 3, 4, 5, 6, 7, 8, 9, 10, 11, 12, 13, 14, 15, 16, 17, 18, 19, 20, 25, 30, 35, 40, 45, or 50 nucleotide substitutions relative to any one of SEQ TD NO s: 7 and 10-16.

The one or more substitutions can be at the 5′ end, the 3′ end, within the nucleotide sequence, or a combination thereof. The one or more substitutions can be contiguous, non-contiguous, or a combination thereof.

TABLE 1
illustrative expression regulatory region sequences

| SEQ ID | | |
|---|---|---|
| NO: | Description | Sequence |
| 1 | CRM8 (single) | GGGGGAGGCTGCTGGTGAATATTAACCAAGGTC |
| | | ACCCCAGTTATCGGAGGAGCAAACAGGGGCTA |
| | | AGTCCAC |
| 2 | 3X CRM8 | GGGGGAGGCTGCTGGTGAATATTAACCAAGGTC |
| | | ACCCCAGTTATCGGAGGAGCAAACAGGGGCTA |
| | | AGTCCACCGGGGGAGGCTGCTGGTGAATATTAA |
| | | CCAAGGTCACCCCAGTTATCGGAGGAGCAAAC |
| | | AGGGGCTAAGTCCACCGGGGGAGGCTGCTGGT |
| | | GAATATTAACCAAGGTCACCCCAGTTATCGGAG |
| | | GAGCAAACAGGGGCTAAGTCCAC |
| 3 | Engineered enhancer | CACTGGGAGGATGTTGAGTAAGATGGAAAACT |
| | (TTRe) based on | ACTGATGACCCTTGCAGAGACAGAGTATTAGGA |
| | murine | CATGTTTGAACAGGGGCCGGGCGATCAGCAGGT |
| | transthyretin | AG |
| | enhancer elements | |
| 4 | Minimal human | CTATTAAGAATATTTCATAGAACGAATGTTCCG |
| | transthyretin | ATGCTCTAATCTCTCTAGACAAGGTTCATATTTG |
| | promoter | TATGGGTTACTTATTCTCTCTTTGTTGACTAAGT |
| | (hTTRm) | CAATAATCAGAATCAGCAGGTTTGCAGTCAGAT |
| | | TGGCAGGGATAAGCAGCCTAGCTCAGGAGAAG |
| | | TGAGTATAAAAGCCCCAGGCTGGGAGCAGCCA |
| | | TC |
| 5 | MVM intron | GTAAGGGTTTAAGGGATGGTTGGTTGGTGGGGT |
| | | ATTAATGTTTAATTACCTGGAGCACCTGCCTGA |
| | | AATCACTTTTTTTCAG |
| 6 | MVM intron with | ACAGAAGTCCACTCATTCTTGAAGAGGTAAGGG |
| | flanking exonic | TTTAAGGGATGGTTGGTTGGTGGGGTATTAATG |
| | sequences | TTTAATTACCTGGAGCACCTGCCTGAAATCACT |
| | | TTTTTTCAGGTTGGACGCGTGTCGACGCCACC |
| 7 | 3xCRM8-mTTRe- | GGGGGAGGCTGCTGGTGAATATTAACCAAGGTC |
| | hTTRm-MVM intron | ACCCCAGTTATCGGAGGAGCAAACAGGGGCTA |
| | (full promoter/ | AGTCCACCGGGGGAGGCTGCTGGTGAATATTAA |
| | enhancer | CCAAGGTCACCCCAGTTATCGGAGGAGCAAAC |
| | sequence) | AGGGGCTAAGTCCACCGGGGGAGGCTGCTGGT |
| | | GAATATTAACCAAGGTCACCCCAGTTATCGGAG |
| | | GAGCAAACAGGGGCTAAGTCCACGGATCCCAC |
| | | TGGGAGGATGTTGAGTAAGATGGAAAACTACT |
| | | GATGACCCTTGCAGAGACAGAGTATTAGGACAT |
| | | GTTTGAACAGGGGCCGGGCGATCAGCAGGTAG |
| | | CTCTAGAGGATCCCCGTCTATTAAGAATATTTC |
| | | ATAGAACGAATGTTCCGATGCTCTAATCTCTCT |
| | | AGACAAGGTTCATATTTGTATGGGTTACTTATTC |
| | | TCTCTTTGTTGACTAAGTCAATAATCAGAATCA |
| | | GCAGGTTTGCAGTCAGATTGGCAGGGATAAGCA |
| | | GCCTAGCTCAGGAGAAGTGAGTATAAAAGCCC |
| | | CAGGCTGGGAGCAGCCATCACAGAAGTCCACTC |
| | | ATTCTTGAAGAGGTAAGGGTTTAAGGGATGGTT |
| | | GGTTGGTGGGGTATTAATGTTTAATTACCTGGA |
| | | GCACCTGCCTGAAATCACTTTTTTTCAGGTTGGA |
| | | CGCGTGTCGACGCCACC |
| 10 | 3xCRM8-mTTRe- | GGGGGAGGCTGCTGGTGAATATTAACCAAGGTC |
| | hTTRm (full | ACCCCAGTTATCGGAGGAGCAAACAGGGGCTA |
| | promoter/enhancer | AGTCCACCGGGGGAGGCTGCTGGTGAATATTAA |
| | sequence without | CCAAGGTCACCCCAGTTATCGGAGGAGCAAAC |
| | MVM intron) | AGGGGCTAAGTCCACCGGGGGAGGCTGCTGGT |
| | | GAATATTAACCAAGGTCACCCCAGTTATCGGAG |
| | | GAGCAAACAGGGGCTAAGTCCACGGATCCCAC |
| | | TGGGAGGATGTTGAGTAAGATGGAAAACTACT |
| | | GATGACCCTTGCAGAGACAGAGTATTAGGACAT |
| | | GTTTGAACAGGGGCCGGGCGATCAGCAGGTAG |
| | | CTCTAGAGGATCCCCGTCTATTAAGAATATTTC |
| | | ATAGAACGAATGTTCCGATGCTCTAATCTCTCT |
| | | AGACAAGGTTCATATTTGTATGGGTTACTTATTC |
| | | TCTCTTTGTTGACTAAGTCAATAATCAGAATCA |
| | | GCAGGTTTGCAGTCAGATTGGCAGGGATAAGCA |
| | | GCCTAGCTCAGGAGAAGTGAGTATAAAAGCCC |
| | | CAGGCTGGGAGCAGCCATC |
| 11 | 1xCRM8-mTTRe- | GGGGGAGGCTGCTGGTGAATATTAACCAAGGTC |
| | hTTRm | ACCCCAGTTATCGGAGGAGCAAACAGGGGCTA |
| | | AGTCCACGGATCCCACTGGGAGGATGTTGAGTA |
| | | AGATGGAAAACTACTGATGACCCTTGCAGAGAC |
| | | AGAGTATTAGGACATGTTTGAACAGGGGCCGG |
| | | GCGATCAGCAGGTAGCTCTAGAGGATCCCCGTC |
| | | TATTAAGAATATTTCATAGAACGAATGTTCCGA |
| | | TGCTCTAATCTCTCTAGACAAGGTTCATATTTGT |
| | | ATGGGTTACTTATTCTCTCTTTGTTGACTAAGTC |
| | | AATAATCAGAATCAGCAGGTTTGCAGTCAGATT |
| | | GGCAGGGATAAGCAGCCTAGCTCAGGAGAAGT |
| | | GAGTATAAAAGCCCCAGGCTGGGAGCAGCCAT |
| | | C |
| 12 | 3xCRM8-mTTRe | GGGGGAGGCTGCTGGTGAATATTAACCAAGGTC |
| | | ACCCCAGTTATCGGAGGAGCAAACAGGGGCTA |
| | | AGTCCACCGGGGGAGGCTGCTGGTGAATATTAA |
| | | CCAAGGTCACCCCAGTTATCGGAGGAGCAAAC |
| | | AGGGGCTAAGTCCACCGGGGGAGGCTGCTGGT |
| | | GAATATTAACCAAGGTCACCCCAGTTATCGGAG |
| | | GAGCAAACAGGGGCTAAGTCCACGGATCCCAC |
| | | TGGGAGGATGTTGAGTAAGATGGAAAACTACT |
| | | GATGACCCTTGCAGAGACAGAGTATTAGGACAT |
| | | GTTTGAACAGGGGCCGGGCGATCAGCAGGTAG |
| 13 | mTTRe-hTTRm | CACTGGGAGGATGTTGAGTAAGATGGAAAACT |
| | | ACTGATGACCCTTGCAGAGACAGAGTATTAGGA |
| | | CATGTTTGAACAGGGGCCGGGCGATCAGCAGGT |
| | | AGCTCTAGAGGATCCCCGTCTATTAAGAATATT |
| | | TCATAGAACGAATGTTCCGATGCTCTAATCTCT |
| | | CTAGACAAGGTTCATATTTGTATGGGTTACTTAT |
| | | TCTCTCTTTGTTGACTAAGTCAATAATCAGAATC |
| | | AGCAGGTTTGCAGTCAGATTGGCAGGGATAAGC |
| | | AGCCTAGCTCAGGAGAAGTGAGTATAAAAGCC |
| | | CCAGGCTGGGAGCAGCCATC |
| 14 | hTTRm-MVM intron | CTATTAAGAATATTTCATAGAACGAATGTTCCG |
| | | ATGCTCTAATCTCTCTAGACAAGGTTCATATTTG |
| | | TATGGGTTACTTATTCTCTCTTTGTTGACTAAGT |
| | | CAATAATCAGAATCAGCAGGTTTGCAGTCAGAT |
| | | TGGCAGGGATAAGCAGCCTAGCTCAGGAGAAG |
| | | TGAGTATAAAAGCCCCAGGCTGGGAGCAGCCA |
| | | TCACAGAAGTCCACTCATTCTTGAAGAGGTAAG |
| | | GGTTTAAGGGATGGTTGGTTGGTGGGGTATTAA |
| | | TGTTTAATTACCTGGAGCACCTGCCTGAAATCA |
| | | CTTTTTTTCAGGTTGGACGCGTGTCGACGCCACC |
| 15 | hTTRm 5′ UTR | ACAGAAGTCCACTCATTCTTG |
| 16 | Kozak | GCCACCATG |

### B. Transgenes

An expression construct disclosed herein can comprise a transgene. The transgene can encode a therapeutic RNA and/or protein. The transgene can encode follistatin.

Follistatin is a monomeric secretory protein encoded by the FST gene. Follistatin (FST) facilitates hypertrophy and/or hyperplasia of skeletal muscle by exhibiting an antagonistic effect on Transforming Growth Factor-β family members including myostatin, which inhibits muscle growth. Follistatin is a myostatin-binding protein that is capable of inactivating myostatin by preventing or inhibiting binding of myostatin to receptors such as ActRITB, thereby reducing downstream signaling. In some cases, follistatin is capable of inhibiting the biosynthesis and secretion of pituitary follicle stimulating hormone (FSH) through binding and inhibition of activin.

Alternative splicing of the approximately 6 kb FST gene precursor mRNA can result in two mature follistatin isoforms, FST288 and FST315. The FST315 isoform results from peptide cleavage of the FST344 splice variant, and the FST288 isoform is produced by cleavage of the FST317 splice variant. FST288 can be a membrane-bound form of follistatin and a potent suppressor of FSH. FST315 can be a serum-circulating isoform that includes a C-terminal acidic region, lower (e.g., 10-fold lower) activin-binding affinity, and reduced potency for FSH inhibition. In some embodiments, an expression construct comprises a transgene that encodes FST344 and/or FST315 to produce a secreted, circulating polypeptide with relatively lower activin binding and FSH inhibition.

A transgene disclosed herein can encode a follistatin polypeptide. A transgene disclosed herein can encode a follistatin polypeptide that comprises, consists essentially of, or consists of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to SEQ ID NO: 9 or SEQ ID NO: 18.

In some embodiments, a transgene encodes a follistatin polypeptide that comprises, consists essentially of, or consists of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity to SEQ ID NO: 9 or SEQ ID NO: 18.

In some embodiments, a transgene encodes a follistatin polypeptide that comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity to SEQ ID NO: 9 or SEQ ID NO: 18.

In some embodiments, a transgene encodes a follistatin polypeptide that comprises the amino acid sequence of SEQ ID NO: 9. In some embodiments, a transgene encodes a follistatin polypeptide that consists essentially of the amino acid sequence of SEQ ID NO: 9. In some embodiments, a transgene encodes a follistatin polypeptide that consists of the amino acid sequence of SEQ ID NO: 9.

In some embodiments, a transgene encodes a follistatin polypeptide that comprises the amino acid sequence of SEQ ID NO: 18. In some embodiments, a transgene encodes a follistatin polypeptide that consists essentially of the amino acid sequence of SEQ ID NO: 18. In some embodiments, a transgene encodes a follistatin polypeptide that consists of the amino acid sequence of SEQ ID NO: 18.

In some embodiments, a transgene encodes a follistatin polypeptide with an amino acid sequence that comprises one or more insertions, deletions, and/or substitutions relative to SEQ ID NO: 9 or SEQ ID NO: 18. For example, the follistatin polypeptide can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to SEQ ID NO: 9 or SEQ ID NO: 18.

In some embodiments, a follistatin polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to SEQ ID NO: 9 or SEQ ID NO: 18.

In some embodiments, a follistatin polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to SEQ ID NO: 9 or SEQ ID NO: 18.

The one or more insertions can be at the N-terminus, the C-terminus, within the amino acid sequence, or a combination thereof. The one or more insertions can be contiguous, non-contiguous, or a combination thereof.

A follistatin polypeptide can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to SEQ ID NO: 9 or SEQ ID NO: 18.

In some embodiments, a follistatin polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to SEQ ID NO: 9 or SEQ ID NO: 18.

In some embodiments, a follistatin polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to SEQ ID NO: 9 or SEQ ID NO: 18.

The one or more deletions can be at the N-terminus, the C-terminus, within the amino acid sequence, or a combination thereof. The one or more deletions can be contiguous, non-contiguous, or a combination thereof.

A follistatin polypeptide can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to SEQ ID NO: 9 or SEQ ID NO: 18.

In some embodiments, a follistatin polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to SEQ ID NO: 9 or SEQ ID NO: 18.

In some embodiments, a follistatin polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to SEQ ID NO: 9 or SEQ ID NO: 18.

The one or more substitutions can be at the N-terminus, the C-terminus, within the amino acid sequence, or a combination thereof. The one or more substitutions can be contiguous, non-contiguous, or a combination thereof. The one or more substitutions can be conservative, non-conservative, or a combination thereof. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser (S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser (S) and Thr (T). A conservative amino acid change can be an amino acid change from one nonpolar amino acid to another nonpolar amino acid. Nonpolar amino acids can include Leu (L), Val (V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one aliphatic amino acid to another aliphatic amino acid. Aliphatic amino acids can include Ala (A), Val (V), Leu (L) and Ile (I). In some embodiments, a conservative amino acid substitution is an amino acid change from one amino acid to another amino acid within one of the following groups: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

Follistatin can comprise one or more domains. For example, follistatin can comprise any one of more of a TB domain (SEQ ID NO: 23), a Follistatin-like 1 domain (SEQ ID NO: 24), a Follistatin-like 2 domain (SEQ ID NO: 25), a Follistatin-like 3 domain (SEQ ID NO: 26), a Kazal-like 1 domain (SEQ ID NO: 27), a Kazal-like 2 domain (SEQ ID NO: 28), a Kazal-like 3 domain (SEQ ID NO: 29), or a variant, functional variant, fragment, or functional fragment thereof.

In some embodiments, a transgene disclosed herein encodes a follistatin polypeptide comprising a domain that comprises, consists essentially of, or consists of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 23-29.

In some embodiments, a transgene disclosed herein encodes a follistatin polypeptide comprising a domain that comprises one or more insertions, deletions, and/or substitutions relative to any one or more of SEQ ID NOs: 23-29.

The follistatin polypeptide can comprise any two or more of SEQ ID NOs: 23-29 or variants, functional variants, fragments, or functional fragments thereof (e.g., comprising at least a minimum amount of sequence identity or sequence similarity as disclosed herein). In some embodiments, the follistatin polypeptide comprises at least 2, at least 3, at least 4, at least 5, or at least six of SEQ ID NOs: 23-29. In some embodiments, the follistatin polypeptide comprises 1, 2, 3, 4, 5, 6, or all 7 of SEQ ID NOs: 23-29. In some embodiments, the follistatin polypeptide comprises SEQ ID NO: 23 or a variant, functional variant, fragment, or functional fragment thereof. In some embodiments, the follistatin polypeptide comprises SEQ ID NO: 24 or a variant, functional variant, fragment, or functional fragment thereof. In some embodiments, the follistatin polypeptide comprises SEQ ID NO: 25 or a variant, functional variant, fragment, or functional fragment thereof. In some embodiments, the follistatin polypeptide comprises SEQ ID NO: 26 or a variant, functional variant, fragment, or functional fragment thereof. In some embodiments, the follistatin polypeptide comprises SEQ ID NO: 27 or a variant, functional variant, fragment, or functional fragment thereof. In some embodiments, the follistatin polypeptide comprises SEQ ID NO: 28 or a variant, functional variant, fragment, or functional fragment thereof. In some embodiments, the follistatin polypeptide comprises SEQ ID NO: 29 or a variant, functional variant, fragment, or functional fragment thereof. In some embodiments, the follistatin polypeptide comprises SEQ ID NO: 23, SEQ ID NO: 24, SEQ ID NO: 25, SEQ ID NO: 26, SEQ ID NO: 27, SEQ ID NO: 28, and SEQ ID NO: 29.

In some embodiments, a transgene of the disclosure that encodes follistatin comprises, consists essentially of, or consists of the nucleotide sequence of SEQ ID NO: 8. In some embodiments, a transgene of the disclosure that encodes follistatin does not comprise or consist of the nucleotide sequence of SEQ ID NO: 8.

In some embodiments, a transgene of the disclosure that encodes follistatin comprises, consists essentially of, or consists of a nucleotide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to SEQ ID NO: 8.

In some embodiments, follistatin encoded by a transgene of the disclosure is an isoform, alternative splicing product, or variant follistatin, for example, any one of SEQ ID NOs: 19-22, or a derivative thereof.

In some embodiments, a transgene disclosed herein encodes a follistatin polypeptide that comprises, consists essentially of, or consists of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 19-22.

In some embodiments, a transgene disclosed herein encodes a follistatin polypeptide that comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity or sequence similarity to any one of SEQ ID NOs: 19-22.

In some embodiments, a transgene encodes a follistatin polypeptide with an amino acid sequence that comprises one or more insertions, deletions, and/or substitutions relative to any one of SEQ ID NOs: 19-22.

In some embodiments, follistatin comprises a signal peptide, e.g., as in SEQ ID NO: 9 and 19. In some embodiments, follistatin lacks a signal peptide, e.g., a mature form of FST after cleavage of the signal peptide, as in SEQ ID NOs: 18 and 20. In some embodiments, an immature form of follistatin comprises the signal peptide of SEQ ID NO: 17. In some embodiments, the signal peptide of SEQ TD NO: 17 is substituted for another suitable signal peptide that can direct secretion and/or membrane insertion of follistatin.

TABLE 2
illustrative transgene sequences

| SEQ ID | | |
|---|---|---|
| NO: | Description | Sequence |
| 8 | FST344 (nucleotide) | ATGGTCCGCGCGAGGCACCAGCCGGGTGGGCTT |
| | | TGCCTCCTGCTGCTGCTGCTCTGCCAGTTCATGG |
| | | AGGACCGCAGTGCCCAGGCTGGGAACTGCTGG |
| | | CTCCGTCAAGCGAAGAACGGCCGCTGCCAGGTC |
| | | CTGTACAAGACCGAACTGAGCAAGGAGGAGTG |
| | | CTGCAGCACCGGCCGGCTGAGCACCTCGTGGAC |
| | | CGAGGAGGACGTGAATGACAACACACTCTTCA |
| | | AGTGGATGATTTTCAACGGGGGCGCCCCCAACT |
| | | GCATCCCCTGTAAAGAAACGTGTGAGAACGTGG |
| | | ACTGTGGACCTGGGAAAAAATGCCGAATGAAC |
| | | AAGAAGAACAAACCCCGCTGCGTCTGCGCCCCG |
| | | GATTGTTCCAACATCACCTGGAAGGGTCCAGTC |
| | | TGCGGGCTGGATGGGAAAACCTACCGCAATGA |
| | | ATGTGCACTCCTAAAGGCAAGATGTAAAGAGC |
| | | AGCCAGAACTGGAAGTCCAGTACCAAGGCAGA |
| | | TGTAAAAAGACTTGTCGGGATGTTTTCTGTCCA |
| | | GGCAGCTCCACATGTGTGGTGGACCAGACCAAT |
| | | AATGCCTACTGTGTGACCTGTAATCGGATTTGC |
| | | CCAGAGCCTGCTTCCTCTGAGCAATATCTCTGT |
| | | GGGAATGATGGAGTCACCTACTCCAGTGCCTGC |
| | | CACCTGAGAAAGGCTACCTGCCTGCTGGGCAGA |
| | | TCCATTGGATTAGCCTATGAGGGAAAGTGTATC |
| | | AAAGCAAAGTCCTGTGAAGATATCCAGTGCACT |
| | | GGTGGGAAAAAATGTTTATGGGATTTCAAGGTT |
| | | GGGAGAGGCCGGTGTTCCCTCTGTGATGAGCTG |
| | | TGCCCTGACAGTAAGTCGGATGAGCCTGTCTGT |
| | | GCCAGTGACAATGCCACTTATGCCAGCGAGTGT |
| | | GCCATGAAGGAAGCTGCCTGCTCCTCAGGTGTG |
| | | CTACTGGAAGTAAAGCACTCCGGATCTTGCAAC |
| | | TCCATTTCGGAAGACACCGAGGAAGAGGAGGA |
| | | AGATGAAGACCAGGACTACAGCTTTCCTATATC |
| | | TTCTATTCTAGAGTGGTAA |
| 9 | FST344 (amino acid) | MVRARHQPGGLCLLLLLLCQFMEDRSAQAGNCW |
| | | LRQAKNGRCQVLYKTELSKEECCSTGRLSTSWTE |
| | | EDVNDNTLFKWMIFNGGAPNCIPCKETCENVDCG |
| | | PGKKCRMNKKNKPRCVCAPDCSNITWKGPVCGL |
| | | DGKTYRNECALLKARCKEQPELEVQYQGRCKKTC |
| | | RDVFCPGSSTCVVDQTNNAYCVTCNRICPEPASS |
| | | EQYLCGNDGVTYSSACHLRKATCLLGRSIGLAYE |
| | | GKCIKAKSCEDIQCTGGKKCLWDFKVGRGRCSLC |
| | | DELCPDSKSDEPVCASDNATYASECAMKEAACSS |
| | | GVLLEVKHSGSCNSISEDTEEEEEDEDQDYSFPI |
| | | SSILEW |
| 17 | FST signal peptide | MVRARHQPGGLCLLLLLLCQFMEDRSAQA |
| 18 | FST315 (FST344 | GNCWLRQAKNGRCQVLYKTELSKEECCSTGRLST |
| | lacking signal peptide) | SWTEEDVNDNTLFKWMIFNGGAPNCIPCKETCEN |
| | | VDCGPGKKCRMNKKNKPRCVCAPDCSNITWKGP |
| | | VCGLDGKTYRNECALLKARCKEQPELEVQYQGR |
| | | CKKTCRDVFCPGSSTCVVDQTNNAYCVTCNRICP |
| | | EPASSEQYLCGNDGVTYSSACHLRKATCLLGRSI |
| | | GLAYEGKCIKAKSCEDIQCTGGKKCLWDFKVGRG |
| | | RCSLCDELCPDSKSDEPVCASDNATYASECAMKE |
| | | AACSSGVLLEVKHSGSCNSISEDTEEEEEDEDQD |
| | | YSFPISSILEW |
| 19 | FST317 (follistatin | MVRARHQPGGLCLLLLLLCQFMEDRSAQAGNCW |
| | alternative splice | LRQAKNGRCQVLYKTELSKEECCSTGRLSTSWTE |
| | product) | EDVNDNTLFKWMIFNGGAPNCIPCKETCENVDCG |
| | | PGKKCRMNKKNKPRCVCAPDCSNITWKGPVCGL |
| | | DGKTYRNECALLKARCKEQPELEVQYQGRCKKTC |
| | | RDVFCPGSSTCVVDQTNNAYCVTCNRICPEPASS |
| | | EQYLCGNDGVTYSSACHLRKATCLLGRSIGLAYE |
| | | GKCIKAKSCEDIQCTGGKKCLWDFKVGRGRCSLC |
| | | DELCPDSKSDEPVCASDNATYASECAMKEAACSS |
| | | GVLLEVKHSGSCN |
| 20 | FST288 (FST317 | GNCWLRQAKNGRCQVLYKTELSKEECCSTGRLST |
| | lacking signal peptide) | SWTEEDVNDNTLFKWMIFNGGAPNCIPCKETCEN |
| | | VDCGPGKKCRMNKKNKPRCVCAPDCSNITWKGP |
| | | VCGLDGKTYRNECALLKARCKEQPELEVQYQGR |
| | | CKKTCRDVFCPGSSTCVVDQTNNAYCVTCNRICP |
| | | EPASSEQYLCGNDGVTYSSACHLRKATCLLGRSIG |
| | | LAYEGKCIKAKSCEDIQCTGGKKCLWDFKVGRGR |
| | | CSLCDELCPDSKSDEPVCASDNATYASECAMKEA |
| | | ACSSGVLLEVKHSGSCN |
| 21 | FST (C56Y variant) | MVRARHQPGGLCLLLLLLCQFMEDRSAQAGNCW |
| | | LRQAKNGRCQVLYKTELSKEECYSTGRLSTSWTE |
| | | EDVNDNTLFKWMIFNGGAPNCIPCKETCENVDCG |
| | | PGKKCRMNKKNKPRCVCAPDCSNITWKGPVCGL |
| | | DGKTYRNECALLKARCKEQPELEVQYQGRCKKTC |
| | | RDVFCPGSSTCVVDQTNNAYCVTCNRICPEPASS |
| | | EQYLCGNDGVTYSSACHLRKATCLLGRSIGLAYE |
| | | GKCIKAKSCEDIQCTGGKKCLWDFKVGRGRCSLC |
| | | DELCPDSKSDEPVCASDNATYASECAMKEAACSS |
| | | GVLLEVKHSGSCNSISEDTEEEEEDEDQDYSFPI |
| | | SSILEW |
| 22 | FST (E152Q variant) | MVRARHQPGGLCLLLLLLCQFMEDRSAQAGNCW |
| | | LRQAKNGRCQVLYKTELSKEECCSTGRLSTSWTE |
| | | EDVNDNTLFKWMIFNGGAPNCIPCKETCENVDCG |
| | | PGKKCRMNKKNKPRCVCAPDCSNITWKGPVCGLD |
| | | GKTYRNECALLKARCKQQPELEVQYQGRCKKTCR |
| | | DVFCPGSSTCVVDQTNNAYCVTCNRICPEPASS |
| | | EQYLCGNDGVTYSSACHLRKATCLLGRSIGLAYE |
| | | GKCIKAKSCEDIQCTGGKKCLWDFKVGRGRCSLC |
| | | DELCPDSKSDEPVCASDNATYASECAMKEAACSS |
| | | GVLLEVKHSGSCNSISEDTEEEEEDEDQDYSFPI |
| | | SSILEW |
| 23 | TB domain of FST | GNCWLRQAKNGRCQVLYKTELSKEECCSTGRLST |
| | | SWTEEDVNDNTLFKWMIFNGGAPNCIPCKETCEN |
| | | VDCGPG |
| 24 | Follistatin-like 1 | TCENVDCGPGKKCRMNKKNKPRCV |
| | domain of FST | |
| 25 | Follistatin-like 2 | TCRDVFCPGSSTCVVDQTNNAYCV |
| | domain of FST | |
| 26 | Follistatin-like 3 | SCEDIQCTGGKKCLWDFKVGRGRCS |
| | domain of FST | |
| 27 | Kazal-like 1 domain of | NKPRCVCAPDCSNITWKGPVCGLDGKTYRNECAL |
| | FST | LKARCKEQPELEVQYQGRCKK |
| 28 | Kazal-like 2 domain of | NAYCVTCNRICPEPASSEQYLCGNDGVTYSSACH |
| | FST | LRKATCLLGRSIGLAYEGKCIK |
| 29 | Kazal-like 3 domain of | RGRCSLCDELCPDSKSDEPVCASDNATYASECAM |
| | FST | KEAACSSGVLLEVKHSGSCNS |

In some embodiments, a transgene (e.g., encoding follistatin) is codon optimized. In some embodiments, a transgene (e.g., encoding follistatin) is a codon-optimized version of a transgene or transgene sequence disclosed herein. Codon optimization can be used to increase expression, e.g., in human cells, such as hepatocytes.

Codon-optimized coding regions can be designed by various different methods, including methods that are published, publicly available, or commercially available. Since the genetic code is degenerate (i.e., each amino acid can be coded by on average three different codons), the DNA sequence can be modified by synonymous nucleotide substitutions without altering the amino acid sequence of the encoded protein.

Illustrative codon optimizing methods are described, e.g., in U.S. Pat. Nos. 7,561,972; 7,561,973; and 7,888,112, and International Patent Application Pub. No. WO 2015/012924, which are incorporated by reference for such disclosure. A transgene sequence that codes for a product (e.g., follistatin) can be modified with synonymous codon sequences. Codon optimization can comprise use of any suitable available codon frequency table, including any disclosed in or referred to in U.S. Pat. Nos. 7,561,972; 7,561,973; and 7,888,112, and International Patent Application Pub. No. WO 2015/012924, which are incorporated by reference for such disclosure. Codons can be selected for a particular tissue or cell type, e.g., hepatocytes.

In some embodiments, the entire length of the open reading frame (ORF) for the product is modified. In some embodiments, only a fragment of the ORF is altered. By using one of these methods, one can apply the codon frequencies to any given polypeptide sequence, and produce a nucleic acid fragment of a codon-optimized coding region which encodes the polypeptide.

The degree of sequence identity between two sequences as disclosed herein can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm. A Needleman and Wunsch global alignment algorithm can be used to align two sequences over their entire length, maximizing the number of matches and minimizes the number of gaps. Default settings can be used.

### II. DELIVERY VECTORS AND PHARMACEUTICAL COMPOSITIONS

Disclosed herein, in some aspects, is a delivery vector that comprises an expression construct. The delivery vector can facilitate delivery of the expression construct to cells, for example, in a cell type-specific or non-cell type specific manner.

A delivery vector can be a non-viral vector. A delivery vector can be a lipid-based vector, for example, a proteo-lipid vehicle (PLV), a lipid nanoparticle (LNP), or a liposome.

A lipid-based vector can comprise an electroneutral lipid. A lipid-based vector can comprise an ionizable lipid. A lipid-based vector can comprise a cationic lipid.

A lipid-based vector can comprise, for example, 1,2-dioleoyl-3-dimethylammonium-propane (DODAP), 1,2-dioleoyl-3-trimethylammonium-propane (DOTAP), 1,2-dioleoyl-sn-glycero-3-phosphoethanolamine (DOPE), Cholesterol, 1,2-dimyristoyl-rac-glycero-3-methoxypolyethylene glycol (DMG-PEG), or any combination thereof.

A delivery vector can comprise a fusogenic protein. A fusogenic protein can be, for example, a reovirus-derived fusion-associated small transmembrane (FAST) protein comprising, for example, an ectodomain, a transmembrane domain, a myristoylation motif, or a combination thereof. A fusogenic protein can be an engineered fusogenic protein. A fusogenic protein can be a fusion protein, for example, comprising a first domain (e.g., myristoylation motif, fusion motif, amphipathic helix, ectodomain, transmembrane domain, and/or endodomain) from a first FAST protein and a second domain (e.g., myristoylation motif, fusion motif, amphipathic helix, ectodomain, transmembrane domain, and/or endodomain) from a second FAST protein. A fusogenic protein can be a fusion protein comprising a first domain from or derived from a p14 FAST protein and a second domain from or derived from a p15 FAST protein.

A delivery vector can be a PLV that comprises a FAST protein, e.g., FAST-PLV. The ability of FAST-PLVs administered locally or systemically to deliver pDNA encoding FST-344, and for gene delivery to effect quantifiable changes in muscle tissue, is shown herein, demonstrating the potential clinical utility of this non-viral delivery platform. In some embodiments, the low immunogenicity of FAST-PLVs is beneficial for this type of gene therapy. FAST-PLV administration can be adjusted to fit a subject's needs. This also allows treatment to be stopped and started as needed. Therapeutic gene expression following systemic FAST-PLV administration can be targeted to specific tissue types by altering the pDNA promoter, which can be utilized in the future to prevent off-target effects.

Compositions, systems, and methods of the disclosure can comprise or utilize delivery vectors, e.g., for delivery of an expression construct disclosed herein.

A delivery vector disclosed herein can be a lipid-based delivery vector (e.g., a proteo-lipid vehicle, PLV). A lipid-based delivery vector disclosed herein can facilitate delivery of an expression construct or polynucleotide disclosed herein, and expression of a transgene of interest after in vivo administration to a subject. For example, a PLV disclosed herein can facilitate expression of a transgene after in vivo administration. A PLV disclosed herein can utilize an effective and re-dosable delivery platform that allows high tolerability compared to alternate formulations or approaches. A PLV can comprise a lipid membrane and/or a lipid bi-layer. A PLV can exclude an enveloped viral vector.

A PLV disclosed herein can comprise one or more (for example, two or more, three or more, four or more, five or more, one, two three, four, five, or six) lipids selected from 1,2-di-O-octadecenyl-3-trimethylammonium propane (DOTMA), 1,2-dioleoyl-3-dimethylammonium-propane (DODAP), 1,2-Dioleyloxy-3-dimethylaminopropane (DODMA), 1,2-dimyristoyl-sn-glycero-3-methoxypolyethylene glycol-2000 (DMG-PEG). 2-dioleoyl-sn-glycero-3-phosphoethanolamine (DOPE) 1,2-dioleoyl-3-trimethylammonium-propane (DOTAP), Dlin-KC2-DMA (KC2), DOBAQ, 18:1 EPC, DDAB, 18:0 EPC, 18:0 DAP, L-a-dioleoyl phosphatidyl choline (DOPC), cholesterol, DF4C11PE (rac-2,3-Di[11-(F-butyl)undecanoyl) glycero-1-phosphoethanolamine), distear-4-ynoyl L-a-phosphatidylethanolamine [DS(9-yne)PE], 18:0 TAP, dipalmitoylphosphatidylcholine (DPPC), phosphatidylcholine (PC), phosphatidic acid (PA), phosphatidylglycerol (PG), phosphatidylethanolamine (PE), phosphatidylserine (PS), distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE), and 2,3-dioleyloxy-N-[2-(sperminecarboxamido)ethyl]-N,N-dimethyl-1-propanaminium trifluoroacetate (DOSPA).

A PLV disclosed herein can comprise one or more ionizable lipids. The charge of ionizable lipids can be dependent upon the pH of the surrounding environment. Ionizable lipids include, but are not limited to, 1,2-dioleoyl-3-dimethylammonium-propane (DODAP), 1,2-dioleoyl-3-trimethylammonium-propane (DOTAP), 1,2-dioleyloxy-3-dimethylaminopropane (DODMA), 1,2-di-O-octadecenyl-3-trimethylammonium propane (DOTMA), DLin-MC3-DMA (MC3), Dlin-KC2-DMA (KC2), DOBAQ, 18:1 EPC, DDAB, 18:0 EPC, 18:0 DAP, and 18:0 TAP.

In some embodiments, ionizable lipids in a PLV disclosed herein comprise, consist essentially of, or consist of DODAP. In some embodiments, ionizable lipids in a PLV disclosed herein comprise, consist essentially of, or consist of DODMA. In some embodiments, ionizable lipids in a PLV disclosed herein comprise, consist essentially of, or consist of DODAP and DODMA. In some embodiments, ionizable lipids (e.g., in a combination or ratio referred to herein) do not include cationic lipids, such as DOTMA and/or DOTAP.

A PLV disclosed herein can comprise one or more cationic lipids. Non-limiting examples of cationic lipids include 1,2-di-O-octadecenyl-3-trimethylammonium propane (DOTMA) and 1,2-dioleoyl-3-trimethylammonium-propane (DOTAP). In some embodiments, cationic lipids are used in a sufficiently low quantity in a PLV to reduce a pro-inflammatory response to the PLV (e.g., Th1 type cytokines or type I interferon) compared to control lipid nanoparticles. In some embodiment, a PLV does not contain or substantially lacks cationic lipids. In some embodiment, a PLV does not contain or substantially lacks DOTAP. In some embodiment, a PLV does not contain or substantially lacks DOTMA. In some embodiment, a PLV does not contain or substantially lacks cationic lipids except for DOTAP. In some embodiment, a PLV does not contain or substantially lacks cationic lipids except for DOTMA. In some embodiment, a PLV does not contain or substantially lacks cationic lipids except for DOTAP and DOTMA. In some embodiments, cationic lipids in a PLV disclosed herein comprise, consist essentially of, or consist of DOTMA. In some embodiments, cationic lipids in a PLV disclosed herein comprise, consist essentially of, or consist of DOTAP. In some embodiments, cationic lipids in a PLV disclosed herein comprise, consist essentially of, or consist of DOTAP and DOTMA.

A PLV disclosed herein can comprise one or more helper lipids. Non-limiting examples of a helper lipids include 2-dioleoyl-sn-glycero-3-phosphoethanolamine (DOPE) and L-a-dioleoyl phosphatidyl choline (DOPC). In some embodiments, helper lipids in a PLV disclosed herein comprise, consist essentially of, or consist of DOPE. In some embodiment, a PLV does not contain or substantially lacks DOPE and/or DOPC.

A PLV disclosed herein can comprise one or more PEGylated lipids. A non-limiting example of a PEGylated lipid is 1,2-dimyristoyl-sn-glycero-3-methoxypolyethylene glycol-2000 (DMG-PEG). In some embodiments, PEGylated lipids in a PLV disclosed herein comprise, consist essentially of, or consist of DMG-PEG. In some embodiment, a PLV does not contain or substantially lacks DMG-PEG.

A PLV disclosed herein can comprise cholesterol. In some embodiment, a PLV does not contain or substantially lacks cholesterol.

In some embodiments, a PLV disclosed herein comprises a combination of lipids at molar ratios appropriate to reduce toxicity, immunogenicity, or a pro-inflammatory response associated with administration of the delivery vector. For example, a PLV can comprise a combination of lipids at molar ratios appropriate to reduce production of pro-inflammatory cytokines, such as tumor necrosis factor alpha (TNF-α), interferon-gamma (IFN-γ), interleukin-6 (TL-6), type I interferon, or a combination thereof associated with administration of the delivery vector. In some embodiments, a PLV disclosed herein comprises a combination of lipids at molar ratios appropriate to reduce complement activation-related pseudoallergy (CARPA). The reduction can be in comparison to, for example, control lipid nanoparticles that comprise a higher proportion of cationic lipids. The reduction can be determined by an experiment in which empty PLV or substantially non-immunogenic cargo is administered (e.g., an expression construct or polynucleotide encoding a reporter, such as GFP). In some embodiments, the combination of lipids in the PLV make the PLV or system more suitable for high dose and/or systemic administration as compared to the control lipid nanoparticles. In some embodiments, a PLV disclosed herein exhibits broader distribution upon systemic administration compared to control lipid nanoparticles or viral vectors. In some embodiments, a PLV disclosed herein exhibits reduced accumulation in the liver upon systemic administration compared to control lipid nanoparticles or viral vectors.

A PLV disclosed herein can exhibit superior properties for delivery of a DNA expression construct or polynucleotide compared to control lipid nanoparticles. For example, in some embodiments a PLV disclosed herein requires less cationic components to neutralize the anionic charge of DNA as compared to control lipid nanoparticles.

In some embodiments, a PLV disclosed herein comprises DODAP. In some embodiments, a PLV disclosed herein comprises DODMA. In some embodiments, a PLV disclosed herein comprises DODAP and DODMA.

In some embodiments, a PLV disclosed herein comprises cationic:ionizable:helper:PEGylated lipids at a molar ratio disclosed herein. The cationic lipid(s) can comprise or consist of DOTAP. The ionizable lipid(s) can comprise or consist of DODAP. The ionizable lipid(s) can comprise or consist of DODMA. The ionizable lipid(s) can comprise or consist of DOTAP. The ionizable lipid(s) can comprise or consist of DODAP and DODMA. The ionizable lipid(s) can comprise or consist of DODAP and DOTAP. The ionizable lipid(s) can comprise or consist of DODMA and DOTAP. The ionizable lipid(s) can comprise or consist of DODAP, DODMA, and DOTAP. The helper lipid(s) can comprise or consist of DOPE. The PEGylated lipid(s) can comprise or consist of DMG-PEG.

In some embodiments, the lipids of a PLV disclosed herein comprise, consist essentially of, or consist of cationic:ionizable:helper:PEGylated lipids at a molar ratio of about 24:42:30:4.

In some embodiments, the lipids of a PLV disclosed herein comprise, consist essentially of, or consist of cationic:ionizable:helper:PEGylated lipids at a molar ratio of about 6:60:30:4.

In some embodiments, the lipids of a PLV disclosed herein comprise, consist essentially of, or consist of cationic:ionizable:helper:PEGylated lipids at a molar ratio of about 0:66:30:4.

In some embodiments, the lipids of a PLV disclosed herein comprise, consist essentially of, or consist of cationic:ionizable:helper:PEGylated lipids at a molar ratio of about 3:63:30:4.

In some embodiments, the lipids of a PLV disclosed herein comprise, consist essentially of, or consist of cationic:ionizable:helper:PEGylated lipids at a molar ratio of about 49.5:24.75:23.75:2.

In some embodiments, the lipids of a PLV disclosed herein comprise, consist essentially of, or consist of cationic:ionizable:helper:PEGylated lipids at a molar ratio of about 49.5:38.5:10:2.

In some embodiments, the lipids of a PLV disclosed herein comprise, consist essentially of, or consist of cationic:ionizable:helper:PEGylated lipids at a molar ratio of about 61.7:26.3:19:3.

In some embodiments, a PLV disclosed herein comprises ionizable, cholesterol, helper, and PEGylated lipids. In some embodiments, the lipids of a PLV disclosed herein comprise, consist essentially of, or consist of ionizable:cholesterol:helper:PEGylated lipids at a molar ratio of about 49.5:38.5:10:2. In some embodiments, the lipids of a PLV disclosed herein comprise, consist essentially of, or consist of ionizable:cholesterol:helper:PEGylated lipids at a molar ratio of about 49.5:24.75:23.75:2. In some embodiments, the lipids of a PLV disclosed herein comprise, consist essentially of, or consist of ionizable:cholesterol:helper:PEGylated lipids at a molar ratio of about 61.7:26.3:19:3.

In some embodiments, the lipids of a PLV disclosed herein comprise, consist essentially of, or consist of DOTAP, DODAP, DOPE and DMG-PEG. In some embodiments, the lipids of a PLV disclosed herein comprise, consist essentially of, or consist of DOTAP, DODAP, DOPE, and DMG-PEG, at a molar ratio or in a mole percentage of about 24:42:30:4.

In some embodiments, the lipids of a PLV disclosed herein comprise, consist essentially of, or consist of DOTAP, DODMA, DOPE and DMG-PEG. In some embodiments, the lipids of a PLV disclosed herein comprise, consist essentially of, or consist of DOTAP, DODMA, DOPE, and DMG-PEG, at a molar ratio or in a mole percentage of about 24:42:30:4.

In some embodiments, the lipids of a PLV disclosed herein comprise, consist essentially of, or consist of DOTAP, DODAP, DODMA, DOPE, and DMG-PEG. In some embodiments, the lipids of a PLV disclosed herein comprise, consist essentially of, or consist of DOTAP, DODAP, DODMA, DOPE, and DMG-PEG, at a molar ratio or in a mole percentage of about 24:21:21:30:4.

In some embodiments, the lipids of a PLV disclosed herein comprise, consist essentially of, or consist of DOTAP, DODAP, DOPE and DMG-PEG. In some embodiments, the lipids of a PLV disclosed herein comprise, consist essentially of, or consist of DOTAP, DODAP, DOPE, and DMG-PEG, at a molar ratio or in a mole percentage of about 6:60:30:4 or 3:63:30:4.

In some embodiments, the lipids of a PLV disclosed herein comprise, consist essentially of, or consist of DODAP, DOPE and DMG-PEG. In some embodiments, the lipids of a PLV disclosed herein comprise, consist essentially of, or consist of DODAP, DOPE, and DMG-PEG, at a molar ratio or in a mole percentage of about 66:30:4.

In some embodiments, the lipids of a PLV disclosed herein comprise, consist essentially of, or consist of DODAP, cholesterol, DOPE, and DMG-PEG. In some embodiments, the lipids of a PLV disclosed herein comprise, consist essentially of, or consist of DODAP, cholesterol, DOPE, and DMG-PEG, at a molar ratio or in a mole percentage of about 49.5:24.75:23.75:2, about 49.5:38.5:10:2, or about 61.7:26.3:19:3.

A delivery vector, such as a PLV, can comprise a fusogenic protein to enhance fusion of the PLV with the plasma membrane of a target cell. Non-limiting examples of fusogenic proteins include a fusion associated small transmembrane (FAST) proteins, herpes simplex virus glycoprotein H, and amphiphilic anionic peptides derived from the N-terminal segment of the HA-2 subunit of influenza virus haemagglutinin, such as the IFN7 and ESCA.

A fusogenic protein can be a fusion associated small transmembrane (FAST) protein or can comprise a functional fragment of a FAST protein. A FAST protein can function receptor-independently, and at physiological pH. In some embodiments, use of a FAST protein in a PLV allows a minimal molar ratio of cationic and/or ionizable lipid to be used in order to neutralize the anionic charge of the nucleic acid, reducing or substantially eliminating the role of ionizable lipid in the delivery process (e.g., endosomal escape). In some embodiments, incorporation of a FAST protein in a PLV enhances intracellular delivery of an expression construct or polynucleotide disclosed herein. In some embodiments, use of a FAST protein in a PLV allows for omission or lower concentrations of cholesterol to be used, for example, compared to control lipid nanoparticles.

The FAST protein family comprises six members named according to their molecular mass in Daltons (p10, p13, p14, p15, p16, and p22).

In some embodiments, a FAST protein utilized in a compositions, system, or method disclosed herein is a native FAST protein found in the family Reoviridae, for example, found in the genus Aquareovirus or Orthoreovirus. Non-limiting examples of orthoreoviruses include BRV (Baboon orthoreovirus), MRV (Mammalian orthoreovirus), NBV (Nelson Bay orthoreovirus), BrRV (Broome orthoreovirus), RRV (Reptilian orthoreovirus), and ARV (Avian orthoreovirus). In some embodiments, a FAST protein utilized in a compositions, system, or method disclosed herein comprises a FAST protein or domain thereof from ARV p10, BrRv p13, RRV p14, BRV p15, AqV p16, or AqV p22.

A FAST protein can comprise an N-terminal ectodomain on the extracellular or external side of the membrane or PLV. The ectodomain can be, for example, about 19-40 residues, with a myristoylation motif, or a myristate moiety on a glycine, such as a penultimate N-terminal glycine. A FAST protein ectodomain can comprise a hydrophobic patch.

A FAST protein can comprise a transmembrane domain that serves as a reverse signal-anchor sequence to direct a bitropic Nₒᵤₜ/Cᵢₙ type I topology in the membrane or PLV.

A FAST protein can comprise a C-terminal endodomain on the cytoplasmic or internal side of the membrane or PLV. A FAST protein endodomain can be, e.g., about 40-140 residues, with a membrane-destabilizing fusion peptide motif. A FAST protein endodomain can comprise a juxtamembrane polybasic motif. A FAST protein endodomain can comprise a membrane-proximal membrane curvature sensor (e.g., an amphipathic alpha helix, such as a helix-kink-helix membrane curvature sensor) to drive pore formation. A FAST protein endodomain can comprise a hydrophobic patch.

A FAST protein can comprise a proline-hinged loop. A FAST protein can comprise a type II polyproline helix. A FAST protein can comprise a conserved region that functions as a fusion peptide, e.g., by promoting rapid lipid bilayer destabilization and membrane merging. A FAST protein can comprise a palmitoylated cysteine residue. A FAST protein can comprise a hydrophobic patch.

Structure-function relationships between different FAST proteins have suggested that overlapping structural motifs of can be exchanged among certain FAST proteins to generate functional chimeric FAST fusion proteins. In some embodiments, a chimeric FAST protein disclosed herein exhibits superior fusion activity compared to a wild-type FAST protein. In some embodiments, FAST-PLVs with chimeric FAST fusion proteins display a favorable toxicity profile while maintaining efficient systemic gene expression by capitalizing on the elegant fusion inducing properties of the Orthoreovirus FAST protein. In some embodiments, incorporation of a chimeric FAST protein into a PLV enhances intracellular delivery and expression of mRNA and pDNA both in vitro and in vivo. These PLVs can also display a favorable immune profile and can be significantly less toxic than conventional LNPs.

Chimeric FAST proteins can be synthesized that combine the domains from different FAST proteins, such the p10, p14, and/or p15 peptides, to form a functional fusogenic protein.

A FAST protein used in a PLV disclosed herein can comprise the ectodomain from the p14 FAST protein or a functional portion thereof, the transmembrane domain from the p14 FAST protein, and the endodomain from the p15 FAST protein or a functional portion thereof. Such a FAST protein can be referred to as a “p14endo15” or “p14e15” FAST protein. In some embodiments, the fusion activity of p14e15 is mediated by the efficient p14 ectodomain fusion peptide and myristate moiety facilitating lipid mixing with the target cell membrane, followed by the p15 endodomain fusion-inducing lipid packing sensor (FLiPs) motif partitioning into the PLV membrane to promote pore formation and liposome-cell fusion activity.

A FAST protein used in a PLV disclosed herein can comprise the ectodomain from the p14 FAST protein or a functional portion thereof, the transmembrane domain from the p15 FAST protein, and the endodomain from the p14 FAST protein or a functional portion thereof. Such a FAST protein can be referred to as “p14TM15”.

A FAST protein used in a PLV disclosed herein can comprise the ectodomain from the p14 FAST protein or a functional portion thereof, the transmembrane domain from the p15 FAST protein, and the endodomain from the p15 FAST protein or a functional portion thereof. Such a FAST protein can be referred to as “p15ecto14” or “p15e14”.

A FAST protein used in a PLV disclosed herein can comprise p10, p13, p14, p15, p16, p22, or a chimeric fusion protein thereof. In some embodiments, the FAST protein is a p14/p15 chimera, p10/p14 chimera, or a p10/p15 chimera. In some embodiments, the FAST protein comprises: (i) the ectodomain and transmembrane domain of p14 and the endodomain of p15; (ii) the ectodomain of p14, and the transmembrane domain and endodomain of p15; or (iii) the ectodomain and endodomain of p14 and the transmembrane of p15.

In some embodiments, a FAST protein comprises an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 30-34.

In some embodiments, a FAST protein comprises an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to at least 40 consecutive amino acids of any one of SEQ ID NOs: 30-34.

TABLE 3 provides illustrative FAST protein sequences.

TABLE 3

| SEQ | | |
|---|---|---|
| ID | | |
| NO | Description | Sequence |
| 30 | P14 | MGSGPSNFVNHAPGEAIVTGLEKGADKVAGTISHTIWEVIAG |
| | | LVALLTFLAFGFWLFKYLQKRRERRRQLTEFQKRYLRNSYRL |
| | | SEIQRPISQHEYEDPYEPPSRRKPPPPPYSTYVNIDNVSAI |
| 31 | P14e15 | MGSGPSNFVNHAPGEAIVTGLEKGADKVAGTISHTIWEVIAG |
| | | LVALLTFLAFGFWLFKYLQWYNRKSKNKKRKEQIREQIELGL |
| | | LSYGAGVASLPLLNVIAHNPGSVISATPIYKGPCTGVPNSRLL |
| | | QITSGTAEENTRILNHDGRNPDGSINV |
| 32 | P14 fusogenic | MGSGPSNFVNHAPGEAIVTGLEKGADKVAGTISHTIWE |
| | protein | |
| | ectodomain | |
| 33 | P15 fusogenic | KLLQWYNRKSKNKKRKEQIREQIELGLLSYGAGVASLPLLN |
| | protein | VIAHNPG |
| | endodomain | |
| 34 | P15 fusogenic | VISATPIYKGPCTGVPNSRLLQITSGTAEENTRILNHDGRNPD |
| | protein | GSINV |
| | endodomain | |

A PLV disclosed herein can comprise one or more ionizable lipids and one or more FAST proteins (e.g., a chimeric FAST protein). In some embodiments, use of a FAST protein in a PLV allows use of certain ionizable lipids and for a favorable ratio of ionizable, helper, and PEGylated lipids.

A delivery vector can comprise a cell penetrating peptide.

A molar ratio of an ionizable lipid to expression construct (e.g., pDNA) can be between about 2.5:1 and about 20:1. In some embodiments, the molar ratio of the ionizable lipid to the expression construct (e.g., pDNA) is about 5:1, about 7.5:1, about 10:1, or about 15:1.

In some embodiments, the molar ratio of the ionizable lipid to the expression construct (e.g., pDNA) is between about 4:1 and about 7.5:1. In some embodiments, the molar ratio of the ionizable lipid to the expression construct (e.g., pDNA) is between about 2.5:1 and about 7:5:1. In some embodiments, the molar ratio of the ionizable lipid to the expression construct (e.g., pDNA) is between about 3:1 and about 7.5:1.

In some embodiments, the molar ratio of the ionizable lipid to the expression construct (e.g., pDNA) is between about 5:1 and about 10:1.

In some embodiments, the molar ratio of the ionizable lipid to the expression construct (e.g., pDNA) is between about 5:1 and about 12:1.

In some embodiments, the molar ratio of the ionizable lipid to the expression construct (e.g., pDNA) is between about 2.5:1 and about 15:1. In some embodiments, the molar ratio of the ionizable lipid to the expression construct (e.g., pDNA) is between about 5:1 and about 15:1. In some embodiments, the molar ratio of the ionizable lipid to the expression construct (e.g., pDNA) is between about 7.5:1 and about 15:1. In some embodiments, the molar ratio of the ionizable lipid to the expression construct (e.g., pDNA) is between about 2.5:1 and about 15:1.

In some embodiments, the molar ratio of the ionizable lipid to the expression construct (e.g., pDNA) is between about 5:1 and about 20:1.

In some embodiments, the molar ratio of the ionizable lipid to the expression construct (e.g., pDNA) is about 5:1.

In some embodiments, the lipids of a PLV disclosed herein comprise, consist essentially of, or consist of DOTAP:DODAP:DOPE:DMG-PEG at a molar ratio or in a mole percentage of about 24:42:30:4, and the molar ratio of ionizable lipid to expression construct (e.g., pDNA) is between about 5:1 and about 10:1.

In some embodiments, the lipids of a PLV disclosed herein comprise, consist essentially of, or consist of DOTAP:DODMA:DOPE:DMG-PEG at a molar ratio or in a mole percentage of about 24:42:30:4, and the molar ratio of ionizable lipid to expression construct (e.g., pDNA) is between about 4:1 and about 7.5:1.

In some embodiments, the lipids of a PLV disclosed herein comprise, consist essentially of, or consist of DOTAP:DODAP:DODMA:DOPE:DMG-PEG at a molar ratio or in a mole percentage of about 24:21:21:30:4, and the molar ratio of ionizable lipid to expression construct (e.g., pDNA) is between about 3:1 to about 7.5:1.

In some embodiments, the lipids of a PLV disclosed herein comprise, consist essentially of, or consist of DOTAP:DODAP:DOPE:DMG-PEG at a molar ratio or in a mole percentage of about 3:63:30:4, and the molar ratio of ionizable lipid to expression construct (e.g., pDNA) is between about 7.5:1 to about 15:1.

In some embodiments, the lipids of a PLV disclosed herein comprise, consist essentially of, or consist of DOTAP:DODAP:DOPE:DMG-PEG at a molar ratio or in a mole percentage of about 3:63:30:4, and the molar ratio of ionizable lipid to expression construct (e.g., pDNA) is between about 5:1 to about 12:1.

In some embodiments, the lipids of a PLV disclosed herein comprise, consist essentially of, or consist of DOTAP:DODAP:DOPE:DMG-PEG at a molar ratio or in a mole percentage of about 6:60:30:4, and the molar ratio of ionizable lipid to expression construct (e.g., pDNA) is between about 5:1 to about 15:1.

In some embodiments, the lipids of a PLV disclosed herein comprise, consist essentially of, or consist of DODAP:DOPE:DMG-PEG at a molar ratio or in a mole percentage of about 66:30:4, and the molar ratio of ionizable lipid to expression construct (e.g., pDNA) is between about 5:1 to about 20:1.

In some embodiments, the lipids of a PLV disclosed herein comprise, consist essentially of, or consist of DODAP:cholesterol:DOPE:DMG-PEG at a molar ratio or in a mole percentage of about 49.5:24.75:23.75:2, and the molar ratio of expression construct (e.g., pDNA) is between about 5:1 to about 15:1.

In some embodiments, the lipids of a PLV disclosed herein comprise, consist essentially of, or consist of DODAP:cholesterol:DOPE:DMG-PEG at a molar ratio or in a mole percentage of about 49.5:38.5:10:2, and the molar ratio of ionizable lipid to expression construct (e.g., pDNA) is between about 5:1 to about 15:1.

In some embodiments, the lipids of a PLV disclosed herein comprise, consist essentially of, or consist of DODAP:cholesterol:DOPE:DMG-PEG at a molar ratio or in a mole percentage of about 61.7:26.3:19:3, and the molar ratio of ionizable lipid expression construct (e.g., pDNA) is between about 5:1 to about 15:1.

In some embodiments, a PLV comprises a vesicle size of less than about 80 nm.

In some embodiments, a PLV is untargeted, and for example, can facilitate delivery of an expression construct to a range of cell types including target cells and non-target cells (e.g., hepatocytes and non-hepatocytes). Specificity of expression in target cells upon non-targeted delivery can be facilitated by an expression regulatory region, such as a cell type-specific promoter.

In some embodiments, a PLV is targeted, for example, can facilitate preferential delivery of an expression construct to a target cell type or population, such as hepatocytes. Specificity of expression in target cells upon targeted delivery can be further enhanced by an expression regulatory region, such as a cell type-specific promoter.

An illustrative method of making a lipid formulation to be used in generating a PLV can comprise heating lipids disclosed herein to 37° C., combining the lipids in ratios disclosed herein, mixing (e.g., vortex mixing), dehydrating the lipid mixture (e.g., in a rotavapor at 60 rpm for 2 hours, under vacuum), rehydrating with 100% ethanol, and sonicating at 37° C.

For generation of a PLV, a NanoAssemblr Benchtop microfluidics mixing instrument can be used to mix organic and aqueous solutions and make the PLVs. The organic solution can comprise or consist of the lipid formulation. The aqueous solution can comprise or consist of nucleic acid cargo, FAST protein (e.g., 5 nM), and acetate buffer (e.g., 10 mM, pH 4.0). The Benchtop NanoAssemblr running protocol can comprise a total flow rate of 12 mL/min and a 3:1 aqueous to organic flow rate ratio. PLVs can be dialyzed in 8000 MWCO dialysis tubing clipped at one end. The loaded tubing can be rinsed with 5 mL of double distilled water and dialyzed in 500 mL of Dialysis Buffer (ENT1844) with gentle stirring (60 rpm) at ambient temperature for 1 hour and repeated twice with fresh Dialysis Buffer. PLVs can be concentrated using a 100 kDa Ultra filter. PLVs can be filter sterilized through 0.2 μm Acrodisc Supor filters.

In some embodiments, a lipid-based delivery vector is or comprises a lipid nanoparticle (LNP). LNPs can be formulated with cationic and/or ionizable lipids that neutralize the anionic charge of nucleic acids and facilitate the endosomal escape of encapsulated nucleic acids through charge-mediated lipid bilayer disruption. LNPs can comprise a combination of different classes of lipids such as cationic or ionizable lipids (CIL), structural lipids (e.g., phospholipid and sterol lipid) and PEG-conjugated lipid (PEG-lipid). These lipids can self-assemble into LNPs under controlled microfluidic mixing with an aqueous phase containing the nucleic acids. PEG-lipids can prevent or reduce aggregation, degradation, and opsonization of the LNPs, while the structural lipids promote the stability and integrity of the nanoparticle.

In some embodiments, an LNP comprises the ionizable lipid DLin-MC3-DMA (MC3). In some embodiments, an LNP comprises DLin-MC3-DMA/DSPC/Cholesterol/PEG-lipid with the molar ratio 50:10:38.5:1.5. In some embodiments a delivery vector is not an LNP.

In some embodiments, a lipid-based delivery vector is or comprises a liposome. A liposome can comprise a cationic lipid, such as a cationic lipid disclosed herein. Illustrative liposomes include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

In some instances, liposomes provided herein also comprise carrier lipids. In some embodiments the carrier lipids are phospholipids. The carrier lipids are optionally any non-phosphate polar lipids. In some instances, liposomes provided herein comprise dipalmitoylphosphatidylcholine (DPPC), phosphatidylcholine (PC; lecithin), phosphatidic acid (PA), phosphatidylglycerol (PG), phosphatidylethanolamine (PE), phosphatidylserine (PS), distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE) and the like, or a combination thereof. In some embodiments, the liposomes further comprise a sterol (e.g., cholesterol) which modulates liposome formation. In some embodiments, a liposome comprises an electroneutral lipid.

In some embodiments, a liposome comprises a cationic lipid. Cationic lipids can have a head group with positive charge (e.g., permanent or substantially permanent positive charge). Non-limiting examples of cationic lipids for use in liposomes include 1,2-di-O-octadecenyl-3-trimethylammonium-propane (DOTMA), 1,2-dioleoyl-3-trimethylammonium-propane (DOTAP), Dimethyldioctadecylammonium bromide (DDAB), and 2,3-dioleyloxy-N-[2-(sperminecarboxamido)ethyl]-N,N-dimethyl-1-propanaminium trifluoroacetate (DOSPA). In some embodiments a delivery vector is not a liposome.

In some embodiments, a lipid-based delivery vector is or comprises a vesicle, micelle or a microsphere. In some embodiments a delivery vector is not a vesicle, a micelle, or a microsphere.

A lipid-based delivery vector can be or comprise a micelle. In some instances, the micelle is a polymeric micelle, characterized by a core shell structure, in which the hydrophobic core is surrounded by a hydrophilic shell. In some cases, the hydrophilic shell further comprises a hydrophilic polymer or copolymer and a pH sensitive component.

Illustrative hydrophilic polymers or copolymers include, but are not limited to, poly(N-substituted acrylamides), poly(N-acryloyl pyrrolidine), poly(N-acryloyl piperidine), poly(N-acryl-L-amino acid amides), poly(ethyl oxazoline), methylcellulose, hydroxypropyl acrylate, hydroxyalkyl cellulose derivatives and poly(vinyl alcohol), poly(N-isopropylacrylamide), poly(N-vinyl-2-pyrrolidone), polyethyleneglycol derivatives, and combinations thereof.

A delivery vector can be or comprise a polymeric micelle exhibiting pH-sensitive properties, e.g., formed by using pH-sensitive polymers including, but not limited to, copolymers from methacrylic acid, methacrylic acid esters and acrylic acid esters, polyvinyl acetate phthalate, hydroxypropyl methyl cellulose phthalate, cellulose acetate phthalate, or cellulose acetate trimellitate.

A delivery vector can comprise a pH-sensitive moiety, which can include, but is not limited to, an alkylacrylic acid such as methacrylic acid, ethylacrylic acid, propyl acrylic acid and butyl acrylic acid, or an amino acid such as glutamic acid.

A delivery vector disclosed herein can be a non-viral vector. In some embodiments, a non-viral vector allows for superior delivery of an expression construct or polynucleotide upon repeat dosing compared to a viral vector, for example, based on reduced immunogenicity.

A delivery vector disclosed herein can be a non-viral, lipid-based delivery vector. A non-viral, lipid-based delivery vector can be, for example, a PLV disclosed herein, a liposome, a lipoplex, a lipid nanoparticle, a vesicle, or a micelle.

In some embodiments, a delivery vector is or comprises a poloxamer, nanoparticle, polyplex, or dendrimer.

A delivery vector can be a nanoparticle, for example, an inorganic nanoparticle, such as a gold, silica, iron oxide, titanium, calcium phosphate, PLGA, poly(ß-amino ester) (PBAE, e.g., PBAE-447), or hydrogel nanoparticle. In some embodiments a delivery vector is not a nanoparticle, e.g., is not an inorganic nanoparticle.

Nucleic acids can be encapsulated in particles through electrostatic association and physical entrapment. To prevent or slow the disassociation of cargo nucleic acids from nanoparticles following systemic administration, a polymerizable conjugate with a degradable, disulfide linkage can be employed. Nanoparticles can be encapsulated with a lipid coating to improve oral bioavailability, minimize enzymatic degradation and cross blood brain barrier. The nanoparticle surface can also be PEGylated to improve water solubility, circulation in vivo, and stealth properties.

A delivery vector can be a polyplex, for example, a complex of one or more polymers and nucleic acids. A polyplex can comprise cationic polymers. Fabrication of a polyplex can be based on self-assembly by ionic interactions. A polyplex can comprise polyethyleneimine, chitosan, poly(beta-amino esters), and/or polyphosphoramidate. In some embodiments a delivery vector is not a polyplex.

A delivery vector can be a dendrimer. A dendrimer can be a highly branched macromolecule with a spherical shape. The surface of dendrimer particles can be functionalized such as, for example, with positive surface charges (cationic dendrimers), which can be employed for the delivery of nucleic acids. Dendrimer-nucleic acid complexes are taken into a cell via endocytosis. In some embodiments a delivery vector is not a dendrimer.

In some embodiments, a delivery vector is or comprises a viral vector, a gamma-retroviral vector, a lentiviral vector, an adenoviral vector, or an adeno-associated viral vector. In some embodiments, a delivery vector is not a viral vector. In some embodiments, a delivery vector is not a retroviral vector. In some embodiments, a delivery vector is not a lentiviral vector. In some embodiments, a delivery vector is not an adenoviral vector. In some embodiments, a delivery vector is not an adeno-associated viral vector.

In some embodiments, a delivery vector is untargeted or is formulated for non-targeted delivery, for example, can facilitate delivery of an expression construct to a range of cell types including target cells and non-target cells (e.g., hepatocytes and non-hepatocytes). Specificity of expression in target cells upon non-targeted delivery can be facilitated by an expression regulatory region, such as a cell type-specific promoter.

In some embodiments, a delivery vector is targeted or is formulated for targeted delivery, for example, can facilitate preferential delivery of an expression construct to a target cell type or population, such as hepatocytes. Specificity of expression in target cells upon targeted delivery can be further enhanced by an expression regulatory region, such as a cell type-specific promoter.

In some embodiments, a vector is a viral vector.

Provided herein, in some aspects, is a pharmaceutical composition that comprises an expression construct or vector disclosed herein and a pharmaceutically-acceptable excipient, carrier, vehicle, or diluent.

### III. METHODS

Among embodiments disclosed herein are compositions and methods suitable for treating a subject. For example, an expression construct, vector, or pharmaceutical composition can be administered to the subject. The subject can be a mammal. The subject can be a human.

In some embodiments, the disclosure provides a method of treating a condition in a subject in need thereof. The condition can be a condition that is alleviated by increased follistatin expression. The condition can be a condition in which follistatin is indicated. The condition can be or can comprise muscle atrophy or muscle wasting, e.g., skeletal or striated muscle atrophy or wasting. The condition can be or can comprise sarcopenia, for example, age-related sarcopenia. The condition can be an aging-related condition. The condition can be a condition associated with cellular senescence or a senescence-associated secretory phenotype (SASP).

The condition can be a condition in which activity of a Transforming Growth Factor-family member contributes to pathogenesis, e.g., a composition or method disclosed herein can inhibit the Transforming Growth Factor-β family member. The condition can be a condition in which activity of myostatin contributes to pathogenesis, e.g., a composition or method disclosed herein can inhibit myostatin. The condition can be a condition in which ActRIIB receptor signaling contributes to pathogenesis, e.g., a composition or method disclosed herein can inhibit ActRIIB receptor signaling. In some embodiments, the condition comprises elevated FSH activity or elevated activin activity, e.g., a composition or method disclosed herein can inhibit FSH or activin.

The condition can be, for example, age-related muscle loss, age-related sarcopenia, alpha-glucosidase deficiency, Becker muscular dystrophy, cachexia, cancer cachexia, Duchenne muscular dystrophy (DMD), facioscapulohumeral muscular dystrophy, inclusion body myositis (IBM), inflammatory myopathy, muscular atrophy, muscular dystrophy, myopathy, myotonic dystrophy, presarcopenia, sarcopenia, severe sarcopenia, spinal muscular atrophy, or sporadic inclusion body myositis (sIBM).

In some embodiments, the condition is an aging-related condition. In some embodiments, the condition is or comprises a fracture, such as a hip fracture. In some embodiments, the condition is osteoporosis.

In some embodiments, an expression construct, vector, or pharmaceutical composition is administered to a subject with limited mobility, for example, a subject that is immobilized. As disclosed herein, compositions and methods that increase muscular size or strength, promote retention of muscular size or strength, or reduce a loss of muscular size or strength, can be useful for, e.g., reducing weakness, improving mobility, reducing the risk of injury, reducing the risk of various complications of immobility, and hastening recovery. In some embodiments, an expression construct, vector, or pharmaceutical composition can be administered to the subject as part of a recovery regimen. For example, an expression construct, vector, or pharmaceutical composition can be administered to a subject recovering from a fracture or dislocation (e.g., a hip fracture). In some embodiments, an expression construct, vector, or pharmaceutical composition is administered to a subject after a surgery, for example, as the subject is recovering from the surgery.

In some embodiments, an expression construct, vector, or pharmaceutical composition is administered to a subject prophylactically. For example, an expression construct, vector, or pharmaceutical composition can be administered to a subject that is at risk (e.g., increased risk) of a fall or injury (e.g., fracture, dislocation, or hip fracture). The subject can be at increased risk, for example, due to a condition such as weakness, disability, unsteady gait, advanced age, or another condition disclosed herein. In some embodiments, an expression construct, vector, or pharmaceutical composition is administered to a subject prior to an expected period of immobilization or reduced mobility. Compositions and methods that increase muscular size or strength, promote retention of muscular size or strength, or reduce a loss of muscular size or strength, can be useful for, e.g., prophylactically reducing weakness or atrophy, prophylactically reducing the risk of injury, prophylactically reducing the risk of various complications of immobility or reduced mobility, and hastening recovery. In some embodiments, an expression construct, vector, or pharmaceutical composition can be administered to the subject as part of a preparatory regimen. For example, an expression construct, vector, or pharmaceutical composition can be administered to a subject prior to a surgery.

In some embodiments, an expression construct, vector, or pharmaceutical composition is administered to a subject at least a day, at least a week, at least two weeks, or at least a month before a surgery or an expected period of immobilization or reduced mobility. In some embodiments, an expression construct, vector, or pharmaceutical composition is administered to a subject about a day, about a week, about two weeks, or about a month before a surgery or an expected period of immobilization or reduced mobility.

The administering can be via any suitable route, for example, local, systemic, topical, parenteral, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal, intracerebral, intraocular, intralesional, intracerebroventricular, intracisternal, or intraparenchymal administration. The administering can be by injection. The administering can be by infusion.

In some embodiments, upon administering the expression construct, vector, or pharmaceutical composition to the subject, strength of a muscle of the subject is increased by at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold relative to before the administering. In some embodiments, the increase in strength is measured after administering the expression construct, vector, or pharmaceutical composition to the subject once (e.g., after a single injection via a systemic route, such as intravenously).

In some embodiments, upon administering the expression construct, vector, or pharmaceutical composition to the subject, strength of a muscle of the subject is increased by at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold relative to a control subject that is not administered the expression construct or is administered a control expression construct, such as an expression construct with a different expression regulatory region (e.g., comprising a CMV promoter). In some embodiments, the increase in strength is measured after administering the expression construct, vector, or pharmaceutical composition to the subject once (e.g., after a single injection via a systemic route, such as intravenously).

In some embodiments, upon administering the expression construct, vector, or pharmaceutical composition to the subject, mass of a muscle of the subject is increased by at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold relative to before the administering. In some embodiments, the muscle mass is measured after administering the expression construct, vector, or pharmaceutical composition to the subject once (e.g., after a single injection via a systemic route, such as intravenously).

In some embodiments, upon administering the expression construct, vector, or pharmaceutical composition to the subject, mass of a muscle of the subject is increased by at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold relative to a control subject that is not administered the expression construct or is administered a control expression construct, such as an expression construct with a different expression regulatory region (e.g., comprising a CMV promoter). In some embodiments, the muscle mass is measured after administering the expression construct, vector, or pharmaceutical composition to the subject once (e.g., after a single injection via a systemic route, such as intravenously).

The muscle can be a skeletal muscle or muscle group. The muscle can be a striated muscle.

In some embodiments, upon administering the expression construct, vector, or pharmaceutical composition to the subject, total muscle mass or total mass of the subject is increased by at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold relative to before the administering. The muscle mass can be total skeletal muscle mass. In some embodiments, the muscle mass or mass is measured after administering the expression construct, vector, or pharmaceutical composition to the subject once (e.g., after a single injection via a systemic route, such as intravenously).

In some embodiments, upon administering the expression construct, vector, or pharmaceutical composition to the subject, total muscle mass or total mass of the subject is increased by at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold relative to a control subject that is not administered the expression construct or is administered a control expression construct, such as an expression construct with a different expression regulatory region (e.g., comprising a CMV promoter). The muscle mass can be total skeletal muscle mass. In some embodiments, the muscle mass or mass is measured after administering the expression construct, vector, or pharmaceutical composition to the subject once (e.g., after a single injection via a systemic route, such as intravenously).

In some embodiments, administering an expression construct, vector, or pharmaceutical composition to the subject results in enhanced retention of muscular strength or mass, or limited loss of muscular strength or mass. For example, the subject can be at risk of losing muscular strength due to immobilization, reduced mobility, a therapeutic intervention that limits mobility or causes reduced strength, or a condition disclosed herein.

In some embodiments, administering the expression construct, vector, or pharmaceutical composition to the subject can limit the loss of muscular strength to no more than about 5%, no more than about 10%, no more than about 15%, no more than about 20%, no more than about 25%, no more than about 30%, no more than about 40%, no more than about 50%, no more than about 60%, no more than about 70%, no more than about 80%, or no more than about 90% relative to before the administering. In some embodiments, the effect on retention of muscular strength is measured after administering the expression construct, vector, or pharmaceutical composition to the subject once (e.g., after a single injection via a systemic route, such as intravenously).

In some embodiments, retention of muscular strength can be at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold better than a control subject that is not administered the expression construct or is administered a control expression construct, such as an expression construct with a different expression regulatory region (e.g., comprising a CMV promoter). In some embodiments, the effect on retention of muscular strength is measured after administering the expression construct, vector, or pharmaceutical composition to the subject once (e.g., after a single injection via a systemic route, such as intravenously).

In some embodiments, administering the expression construct, vector, or pharmaceutical composition to the subject can limit the loss of mass of a muscle to no more than about 5%, no more than about 10%, no more than about 15%, no more than about 20%, no more than about 25%, no more than about 30%, no more than about 40%, no more than about 50%, no more than about 60%, no more than about 70%, no more than about 80%, or no more than about 90% relative to before the administering. In some embodiments, the loss of mass of the muscle is measured after administering the expression construct, vector, or pharmaceutical composition to the subject once (e.g., after a single injection via a systemic route, such as intravenously).

In some embodiments, retention mass of a muscle can be at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold better than a control subject that is not administered the expression construct or is administered a control expression construct, such as an expression construct with a different expression regulatory region (e.g., comprising a CMV promoter). In some embodiments, the effect on retention of mass of the muscle is measured after administering the expression construct, vector, or pharmaceutical composition to the subject once (e.g., after a single injection via a systemic route, such as intravenously).

In some embodiments, administering the expression construct, vector, or pharmaceutical composition to the subject can limit the loss of total muscle mass to no more than about 5%, no more than about 10%, no more than about 15%, no more than about 20%, no more than about 25%, no more than about 30%, no more than about 40%, no more than about 50%, no more than about 60%, no more than about 70%, no more than about 80%, or no more than about 90% relative to before the administering. In some embodiments, the effect on loss of total muscle mass is measured after administering the expression construct, vector, or pharmaceutical composition to the subject once (e.g., after a single injection via a systemic route, such as intravenously).

In some embodiments, retention of total muscle mass can be at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold better than a control subject that is not administered the expression construct or is administered a control expression construct, such as an expression construct with a different expression regulatory region (e.g., comprising a CMV promoter). In some embodiments, the effect on retention of total muscle mass is measured after administering the expression construct, vector, or pharmaceutical composition to the subject once (e.g., after a single injection via a systemic route, such as intravenously).

A change (e.g., increase) in muscle mass can be determined using any suitable method and/or apparatus, for example, via imaging (e.g., MRI or CT), 24-hour urinary creatinine method, Dual-Energy X-Ray Absorptiometry (DEXA), Total Body Potassium (TBK), Total Body Electrical Conductivity (TOBEC), or Wheat germ agglutinin staining of muscle tissue. The muscle mass can be, e.g., skeletal muscle or straited muscle.

Strength and changes in strength can be determined using any suitable method, apparatus, or scale, for example, a grip strength meter test (e.g., dynamometer), the Oxford Scale, a functional test, a mechanical test, or a manual test.

A muscle or muscle group evaluated in a test of muscle strength or mass can include, without limitation, shoulder abductors, elbow flexors, elbow extensors, wrist extensors, finger flexors, hand intrinsics, hip flexors, knee extensors, dorsiflexors, great toe extensor, plantar flexors, biceps, triceps, quadriceps, abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, biceps brachii, biceps femoris, brachialis, brachioradialis, coracobrachialis, deltoid, diaphragm, erector spinae, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, gastrocnemius, gemellus inferior, gemellus superior, gluteus maximus, gluteus medius, gluteus minimus, gracilis, iliacus, infraspinatus, infraspinatus, internal oblique abdominis, interossei—dorsal of foot, interossei—dorsal of hand, interossei—palmar of hand, interossei—plantar of foot, interspinales, intertransversarii, latissimus dorsi, latissimus dorsi, levator scapulae, levatores costarum, lumbricals of foot, lumbricals of hand, obliquus capitis inferior, obliquus capitis superior, obturator externus, obturator internus, opponens digiti minimi (hand), opponens pollicis, palmaris brevis, palmaris longus, pectineus, pectoralis major, pectoralis minor, peroneus brevis, peroneus longus, peroneus tertius, piriformis, plantaris, popliteus, pronator quadratus, pronator teres, psoas major, psoas minor, pyramidalis, quadratus femoris, quadratus lumborum, quadratus plantae, rectus abdominis, rectus capitus posterior major, rectus capitus posterior minor, rectus femoris, rhomboid major, rhomboid minor, sartorius, semimembranosus, semitendinosus, serratus anterior, serratus posterior inferior, serratus posterior superior, soleus, splenius capitis, splenius cervicis, subscapularis, supinator, supraspinatus, supraspinatus, tensor fasciae lata, teres minor, tibialis anterior, tibialis posterior, transversospinalis, transversus abdominis, trapezius, triceps, vastus intermedius, vastus lateralis, vastus medialis, or any combination thereof.

An expression construct, vector, pharmaceutical composition, or method disclosed herein can elicit improved performance of a subject in a functional test, or can limit a reduction in performance of a subject in a functional test. For example, in some embodiments a distance walked by a subject in a walk test, such as a 6 minute walk test or a shuttle walk test, is increased. In some embodiments, upon administering the expression construct, vector, or pharmaceutical composition to the subject, a distance walked by the subject in a walk test (e.g., a 6 minute walk test) is increased by at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold relative to before the administering. In some embodiments, the effect is measured after administering the expression construct, vector, or pharmaceutical composition to the subject once (e.g., after a single injection via a systemic route, such as intravenously).

In some embodiments, upon administering the expression construct, vector, or pharmaceutical composition to the subject, a distance walked by the subject in a walk test (e.g., a 6 minute walk test) is at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold greater than a control subject that is not administered the expression construct or is administered a control expression construct, such as an expression construct with a different expression regulatory region (e.g., comprising a CMV promoter). In some embodiments, the effect is measured after administering the expression construct, vector, or pharmaceutical composition to the subject once (e.g., after a single injection via a systemic route, such as intravenously).

A walk test, such as a 6 minute walk test, can be a sub-maximal exercise test used to assess, e.g., functional or muscular capacity and endurance. The distance covered over a time of 6 minutes can be used as the outcome by which to compare changes in performance capacity. A six minute walk test can be as described in, e.g., ATS Committee on Proficiency Standards for Clinical Pulmonary Function Laboratories. “ATS statement: guidelines for the six-minute walk test.” Am J Respir Crit Care Med 166 (2002): 111-117.

An effect, for example, a change in strength of a muscle, mass of a muscle, total muscle mass of a subject, or performance in a functional test, can be determined any suitable amount of time after administering the expression construct, vector, or pharmaceutical composition to the subject. In some embodiments, the effect is measured about 2 weeks, about 3 weeks, about 4 weeks, about 6 weeks, about 8 weeks, about 10 weeks, about 12 weeks, about 14 weeks, about 16 weeks, about 20 weeks, about 26 weeks, about 8 months, about 10 months, about 12 months, about 18 months, about 24 months, or about 30 months after administering the expression construct, vector, or pharmaceutical composition to the subject. In some embodiments, the effect is measured after administering the expression construct, vector, or pharmaceutical composition to the subject once (e.g., after a single injection via a systemic route, such as intravenously).

In some embodiments, an effect disclosed herein, for example, a change in strength of a muscle, mass of a muscle, total muscle mass of a subject, or performance in a functional test, persists (e.g., remains significant) for at least about 2 weeks, at least about 3 weeks, at least about 4 weeks, at least about 6 weeks, at least about 8 weeks, at least about 10 weeks, at least about 12 weeks, at least about 14 weeks, at least about 16 weeks, at least about 20 weeks, at least about 26 weeks, at least about 8 months, at least about 10 months, at least about 12 months, at least about 18 months, at least about 24 months, or at least about 30 months after administering the expression construct, vector, or pharmaceutical composition to the subject. In some embodiments, the effect is measured after administering the expression construct, vector, or pharmaceutical composition to the subject once (e.g., after a single injection via a systemic route, such as intravenously).

In some embodiments, administering the expression construct, vector, or pharmaceutical composition of the disclosure results in an effect disclosed herein, for example, a change in strength of a muscle, mass of a muscle, total muscle mass of a subject, or performance in a functional test, that persists for at least about 5 days, at least about a week, least about 2 weeks, at least about 3 weeks, at least about 4 weeks, at least about 6 weeks, at least about 8 weeks, at least about 10 weeks, at least about 12 weeks, at least about 14 weeks, at least about 16 weeks, at least about 20 weeks, at least about 26 weeks, at least about 8 months, at least about 10 months, or at least about 12 months longer than an effect resulting from administering a control expression construct, vector, or pharmaceutical composition, such as an expression construct with a different expression regulatory region (e.g., comprising a CMV promoter).

Effects disclosed herein (e.g., average effects) can be determined by evaluating one or more cohorts of subjects. In some embodiments, an effect disclosed herein is determined by evaluating a cohort of subjects administered an expression construct, and determining an average effect in the cohort of subjects, e.g., compared to a cohort of control subjects that are administered a control expression construct, such as an expression construct with a different expression regulatory region (e.g., comprising a CMV promoter).

### IV. EMBODIMENTS

Embodiment 1. An expression construct comprising an expression regulatory region operatively linked to a transgene that encodes follistatin, wherein the expression regulatory region comprises, from 5′ to 3′: (i) a CRM8 element and an engineered transthyretin enhancer (TTRe); or (ii) the TTRe and a human transthyretin promoter (hTTRm).

Embodiment 2. The expression construct of embodiment 1, wherein the expression regulatory region comprises, from 5′ to 3′: the CRM8 element, the TTRe, and the hTTRm.

Embodiment 3. The expression construct of embodiment 1 or embodiment 2, wherein the hTTRm comprises a nucleotide sequence with at least 90% sequence identity to SEQ ID NO: 4.

Embodiment 4. An expression construct comprising an expression regulatory region operatively linked to a transgene that encodes follistatin, wherein the expression regulatory region comprises a human transthyretin promoter (hTTRm) nucleotide sequence with at least 90% sequence identity to SEQ ID NO: 4.

Embodiment 5. The expression construct of embodiment 4, wherein the expression regulatory region further comprises a CRM8 element.

Embodiment 6. The expression construct of embodiment 4 or embodiment 5, wherein the expression regulatory region further comprises an engineered transthyretin enhancer (TTRe).

Embodiment 7. The expression construct of any one of embodiments 1-6, wherein the expression regulatory region further comprises an intron.

Embodiment 8. The expression construct of embodiment 7, wherein the intron is an MVM intron.

Embodiment 9. The expression construct of any one of embodiments 1-3 or 5-8, wherein the CRM8 element comprises a nucleotide sequence with at least 90% sequence identity to SEQ ID NO: 1 or SEQ ID NO: 2.

Embodiment 10. The expression construct of any one of embodiments 1-3 or 5-9, wherein the CRM8 element comprises a nucleotide sequence that is SEQ ID NO: 1 or SEQ ID NO: 2.

Embodiment 11. The expression construct of any one of embodiments 1-3 or 6-10, wherein the TTRe comprises a nucleotide sequence with at least 90% sequence identity to SEQ ID NO: 3.

Embodiment 12. The expression construct of any one of embodiments 1-3 or 6-11, wherein the TTRe comprises a nucleotide sequence that is SEQ ID NO: 3.

Embodiment 13. The expression construct of any one of embodiments 1-12, wherein the hTTRm comprises a nucleotide sequence that is SEQ ID NO: 4.

Embodiment 14. The expression construct of any one of embodiments 7-13, wherein the intron comprises a nucleotide sequence with at least 90% sequence identity to SEQ ID NO: 5.

Embodiment 15. The expression construct of any one of embodiments 7-14, wherein the intron comprises a nucleotide sequence that is SEQ ID NO: 5.

Embodiment 16. The expression construct of any one of embodiments 1-15, wherein the expression construct further comprises a hTTRm 5′ UTR.

Embodiment 17. The expression construct of embodiment 16, wherein the hTTRm 5′ UTR comprises the nucleotide sequence of SEQ ID NO: 15.

Embodiment 18. The expression construct of any one of embodiments 1-17, wherein the expression regulatory region comprises a nucleotide sequence with at least 90% sequence identity to SEQ ID NO: 11.

Embodiment 19. The expression construct of any one of embodiments 1-18, wherein the expression regulatory region comprises a nucleotide sequence with at least 95% sequence identity to SEQ ID NO: 11.

Embodiment 20. The expression construct of any one of embodiments 1-19, wherein the expression regulatory region comprises a nucleotide sequence that is SEQ ID NO: 11.

Embodiment 21. The expression construct of any one of embodiments 1-20, wherein the expression regulatory region comprises a nucleotide sequence with at least 90% sequence identity to SEQ ID NO: 7.

Embodiment 22. The expression construct of any one of embodiments 1-21, wherein the expression regulatory region comprises a nucleotide sequence with at least 95% sequence identity to SEQ ID NO: 7.

Embodiment 23. The expression construct of any one of embodiments 1-22, wherein the expression regulatory region comprises a nucleotide sequence that is SEQ ID NO: 7.

Embodiment 24. An expression construct comprising an expression regulatory region that comprises a nucleotide sequence with at least 95% sequence identity to SEQ ID NO: 11.

Embodiment 25. The expression construct of embodiment 24, wherein the expression regulatory region comprises a nucleotide sequence with at least 98% sequence identity to SEQ ID NO: 11.

Embodiment 26. The expression construct of embodiment 24 or embodiment 25, wherein the expression regulatory region comprises a nucleotide sequence that is SEQ ID NO: 11.

Embodiment 27. An expression construct comprising an expression regulatory region that comprises a nucleotide sequence with at least 90% sequence identity to SEQ ID NO: 7.

Embodiment 28. The expression construct of embodiment 27, wherein the expression regulatory region comprises a nucleotide sequence with at least 98% sequence identity to SEQ ID NO: 7.

Embodiment 29. The expression construct of embodiment 27 or embodiment 28, wherein the expression regulatory region comprises a nucleotide sequence that is SEQ ID NO: 7.

Embodiment 30. The expression construct of any one of embodiments 24-29, wherein the expression regulatory region is operatively linked to a transgene.

Embodiment 31. The expression construct of embodiment 30, wherein the transgene encodes a therapeutic RNA or protein.

Embodiment 32. The expression construct of embodiment 30 or embodiment 31, wherein the transgene encodes follistatin.

Embodiment 33. The expression construct of any one of embodiments 1-23 and 32, wherein the follistatin comprises an amino acid sequence with at least 90% sequence identity to any one of SEQ ID NOs: 23-29.

Embodiment 34. The expression construct of any one of embodiments 1-23 and 32-33, wherein the follistatin comprises an amino acid sequence that is any one of SEQ ID NOs: 23-29.

Embodiment 35. The expression construct of any one of embodiments 1-23 and 32-34, wherein the follistatin comprises the amino acid sequences of each of SEQ ID NOs: 23-29.

Embodiment 36. The expression construct of any one of embodiments 1-23 and 32-35, wherein the follistatin comprises an amino acid sequence with at least 90% sequence identity to SEQ ID NO: 9 or SEQ ID NO: 18.

Embodiment 37. The expression construct of any one of embodiments 1-23 and 32-36, wherein the follistatin comprises an amino acid sequence with at least 95% sequence identity to SEQ ID NO: 9 or SEQ ID NO: 18.

Embodiment 38. The expression construct of any one of embodiments 1-23 and 32-37, wherein the follistatin comprises an amino acid sequence that is SEQ ID NO: 9 or SEQ ID NO: 18.

Embodiment 39. The expression construct of any one of embodiments 1-23 and 32-38, wherein the follistatin comprises an amino acid sequence with at least 90% sequence identity to any one of SEQ ID NOs: 19-22.

Embodiment 40. The expression construct of any one of embodiments 1-39, wherein the transgene comprises a nucleotide sequence with at least 90% sequence identity to SEQ ID NO: 8.

Embodiment 41. The expression construct of any one of embodiments 1-40, wherein the transgene comprises a nucleotide sequence that is SEQ ID NO: 8.

Embodiment 42. The expression construct of any one of embodiments 1-41, wherein the expression construct is a DNA plasmid, nanoplasmid, or minicircle.

Embodiment 43. A vector comprising the expression construct of any one of embodiments 1-42.

Embodiment 44. The vector of embodiment 43, wherein the vector is a non-viral vector.

Embodiment 45. The vector of embodiment 43 or embodiment 44, wherein the vector is a lipid-based vector.

Embodiment 46. The vector of any one of embodiments 43-45, wherein the vector is a proteo-lipid vehicle (PLV), a lipid nanoparticle (LNP), or a liposome.

Embodiment 47. The vector of any one of embodiments 43-46, wherein the vector comprises a fusogenic protein.

Embodiment 48. The vector of any one of embodiments 43-45, wherein the vector is a PLV comprising an ionizable lipid and a fusion associated small transmembrane (FAST) protein.

Embodiment 49. A cell comprising the expression construct of any one of embodiments 1-42.

Embodiment 50. A pharmaceutical composition comprising the expression construct of any one of embodiments 1-42 or the vector of any one of embodiments 43-48, and a pharmaceutically-acceptable excipient, carrier, vehicle, or diluent.

Embodiment 51. A method of treating a condition in a subject in need thereof, the method comprising administering to the subject an effective amount of the expression construct of any one of embodiments 1-42, the vector of any one of embodiments 43-48, the cell of embodiment 49, or the pharmaceutical composition of embodiment 50, thereby treating the condition.

Embodiment 52. A method of increasing muscle mass or muscle strength in a subject, the method comprising administering to the subject an effective amount of the expression construct of any one of embodiments 1-42, the vector of any one of embodiments 43-48, the cell of embodiment 49, or the pharmaceutical composition of embodiment 50, thereby increasing the muscle mass or muscle strength in the subject.

Embodiment 53. A method of promoting retention of muscle mass or muscle strength in a subject, the method comprising administering to the subject an effective amount of the expression construct of any one of embodiments 1-42, the vector of any one of embodiments 43-48, the cell of embodiment 49, or the pharmaceutical composition of embodiment 50, thereby promoting the retention of muscle mass or muscle strength in the subject.

Embodiment 54. A method of reducing loss of muscle mass or muscle strength in a subject, the method comprising administering to the subject an effective amount of the expression construct of any one of embodiments 1-42, the vector of any one of embodiments 43-48, the cell of embodiment 49, or the pharmaceutical composition of embodiment 50, thereby reducing the loss of muscle mass or muscle strength in the subject.

Embodiment 55. A method comprising administering an effective amount of the expression construct of any one of embodiments 1-42, the vector of any one of embodiments 43-48, the cell of embodiment 49, or the pharmaceutical composition of embodiment 50 to a subject prior to a surgery, a period of immobilization, or a period of reduced mobility.

Embodiment 56. A method of increasing muscle mass or strength in a subject, the method comprising administering to the subject an effective amount of an expression construct comprising an expression regulatory region operatively linked to a transgene that encodes follistatin, wherein the expression regulatory region induces preferential expression of the transgene in liver tissue, thereby increasing the muscle mass or muscle strength in the subject.

Embodiment 57. A method of promoting retention of muscle mass or muscle strength in a subject, the method comprising administering to the subject an effective amount of an expression construct comprising an expression regulatory region operatively linked to a transgene that encodes follistatin, wherein the expression regulatory region induces preferential expression of the transgene in liver tissue, thereby promoting the retention of muscle mass or muscle strength in the subject.

Embodiment 58. A method of reducing loss of muscle mass or muscle strength in a subject, the method comprising administering to the subject an effective amount of an expression construct comprising an expression regulatory region operatively linked to a transgene that encodes follistatin, wherein the expression regulatory region induces preferential expression of the transgene in liver tissue, thereby reducing the loss of muscle mass or muscle strength in the subject.

Embodiment 59. A method comprising administering an effective amount of an expression construct to a subject prior to a surgery, a period of immobilization, or a period of reduced mobility, wherein the expression construct comprises an expression regulatory region operatively linked to a transgene that encodes follistatin, wherein the expression regulatory region induces preferential expression of the transgene in liver tissue.

Embodiment 60. The method of any one of embodiments 51-59, wherein the administering is parenteral.

Embodiment 61. The method of any one of embodiments 51-60, wherein the administering is systemic.

Embodiment 62. The method of any one of embodiments 51-61, wherein the administering is intravenous.

Embodiment 63. The method of any one of embodiments 51-62, wherein upon the administering to the subject, strength of a skeletal muscle of the subject is increased by at least 5% relative to before the administering.

Embodiment 64. The method of any one of embodiments 51-63, wherein upon the administering to the subject, strength of a skeletal muscle of the subject is increased by at least 5% relative to a control subject that is not administered the expression construct or is administered a control expression construct.

Embodiment 65. The method of embodiment 63 or embodiment 64, wherein the strength of the skeletal muscle is as determined by a grip strength meter test.

Embodiment 66. The method of any one of embodiments 51-65, wherein upon the administering to the subject, mass of a skeletal muscle of the subject is increased by at least 5% relative to before the administering.

Embodiment 67. The method of any one of embodiments 51-66, wherein upon the administering to the subject, mass of a skeletal muscle of the subject is increased by at least 5% relative to a control subject that is not administered the expression construct or is administered a control expression construct.

Embodiment 68. The method of embodiment 66 or embodiment 67, wherein the increase in muscle mass is as determined by imaging or Wheat germ agglutinin staining of muscle tissue.

Embodiment 69. The method of any one of embodiments 64-68, wherein the control expression construct comprises a CMV promoter that drives expression of follistatin.

Embodiment 70. The method of any one of embodiments 51-69, wherein the subject is a mammal.

Embodiment 71. The method of any one of embodiments 51-70, wherein the subject is a human.

Embodiment 72. The method of embodiment 51, wherein the condition is or comprises age-related muscle loss, alpha-glucosidase deficiency, Becker muscular dystrophy, Duchenne muscular dystrophy (DMD), facioscapulohumeral muscular dystrophy, inclusion body myositis (IBM), inflammatory myopathy, muscular atrophy, muscular dystrophy, myopathy, myotonic dystrophy, sarcopenia, or sporadic inclusion body or myositis (sIBM).

Embodiment 73. The method of embodiment 56, wherein the expression construct is encapsulated in a proteo-lipid vehicle (PLV) that comprises DOTAP:DODAP:DOPE:DMG-PEG at a mole percentage of 3:63:30:4.

Embodiment 74. The vector of embodiment 46 or 47, wherein the vector comprises the PLV, wherein the PLV comprises DOTAP:DODAP:DOPE:DMG-PEG at a mole percentage of 3:63:30:4.

### V. EXAMPLES

The following examples are included for illustrative purposes only and are not intended to limit the scope of the disclosure.

### C. Example 1: Expression Construct Design

Constructs were designed for expression of the protein follistatin (FST), for use in a gene therapy approach to elevate FST expression.

A promoter/enhancer sequence was designed comprising, from 5′ to 3′: (i) three copies of a human CRM8 enhancer (part of the Serpinal promoter sequence, SEQ ID NO: 1), (ii) an engineered enhancer based on murine transthyretin enhancer elements (mTTRe, SEQ ID NO: 3), (iii) a minimal human transthyretin promoter (hTTRm, SEQ ID NO: 4), and (iv) an MVM intron (SEQ ID NO: 5). The promoter/enhancer also included a hTTRm 5′ UTR and a Kozak sequence, and some spacer nucleotides. The full sequence of the promoter/enhancer is provided in SEQ ID NO: 7. The promoter was used to drive expression of follistatin (FST or FST344, SEQ ID NO: 9, encoded by SEQ ID NO: 8).

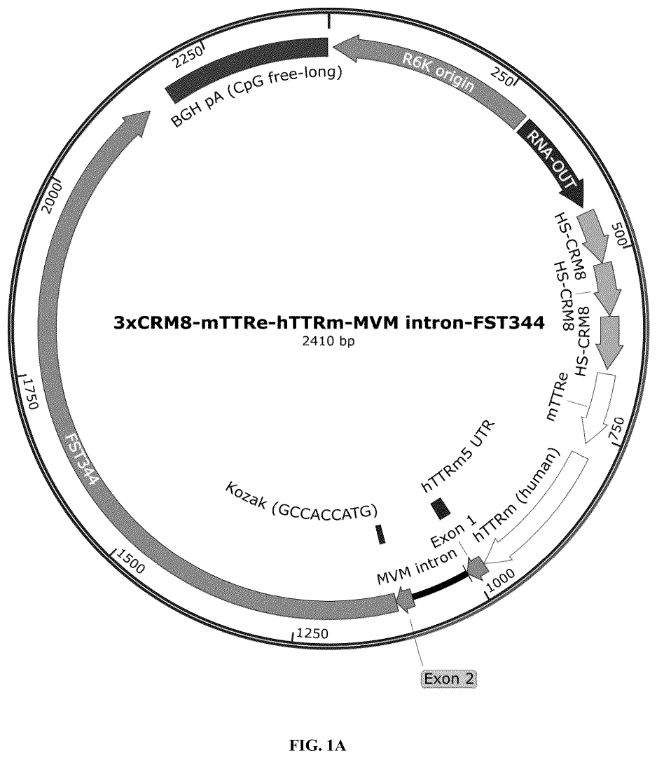

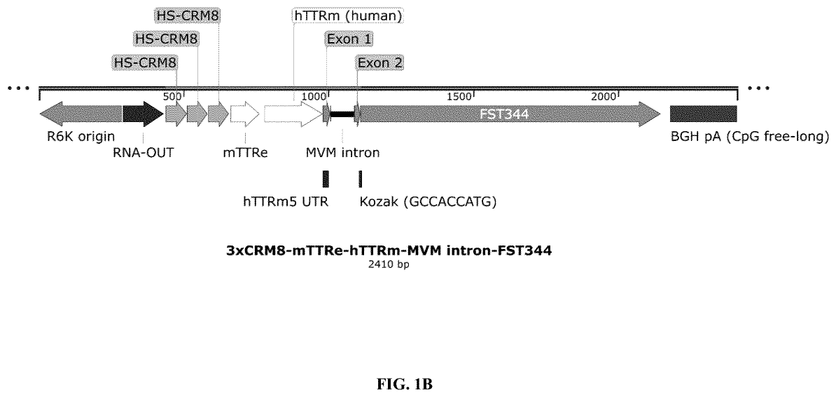

The promoter/enhancer DNA sequence and FST344-encoding DNA sequence were cloned into an a nanoplasmid. Circular and linear maps of the plasmid are provided in FIG. 1A and FIG. 1B, respectively.

Additional expression constructs were produced with expression of FST driven by a CMV promoter, an ApoE promoter, or a SerpEnh promoter.

### D. Example 2: Induction of Follistatin Expression In Vitro

This example demonstrates induction of follistatin (FST) expression in myoblasts using an expression construct disclosed herein. FST can facilitate hypertrophy of skeletal muscle by exhibiting an antagonistic effect on myostatin—a member of the Transforming Growth Factor-β family that inhibits muscle growth. Experiments were undertaken to determine if delivery of pDNA encoding the FST-344 splice variant in FAST-PLV would be a viable alternative to AAV-based therapies.

C2C12 mouse myoblasts were cultured in high glucose-DMEM with 10% FBS and 1% penicillin/streptomycin. 20,000-40,000 cells were seeded per well in 48-well tissue-culture treated plates and left overnight. The cells were treated for 24-72 hours treated with 1000 ng of pDNA expression construct encoding FST driven by a CMV promoter. The pDNA was delivered using proteo-lipid vehicles (PLVs). As a control, cells were treated with PLVs containing DNA encoding GFP driven by the CMV promoter.

Expression of FST and phosphorylation of Akt and mTOR were quantified by Western Blot. Mouse myoblasts (C2C12) were purchased from ATCC (Manassas, VA) and cultured in high glucose-DMEM with 10% FBS and 1% penicillin/streptomycin. Cells were lysed in ice-cold Pierce RIPA buffer. Protein amount was determined using Pierce BCA protein assay. Equal amounts of total protein from each lysate were loaded onto Mini-PROTEAN 4-20% Gradient TGX precast gels. Separated protein was transferred to nitrocellulose membranes. Membranes were blocked with fluorescent western blocking buffer for 1 hour at room temperature. Primary antibodies were diluted 1:1000 in blocking buffer and added to the membranes overnight at 4° C. with shaking. Goat anti-rabbit Alexa Fluor 680, donkey anti-goat Alexa Fluor 680, or goat anti-mouse Alexa Fluor 750 were diluted 1:10000 in blocking buffer and added for 1 hour at room temperature in the dark. Membranes were visualized on the LI-COR Odyssey. Goat anti-follistatin antibody (AF669) was purchased from R&D systems (Minneapolis, United States). Phospho-Akt Ser473 (4069), pan-Akt (2920), phosphor-mTOR Ser2448 (2971), and mTOR (2972) antibodies were purchased from Cell Signaling Technology (Danvers, United States). Anti-GAPDH antibody (ab8245) was purchased from Abcam (Cambridge, United Kingdom).

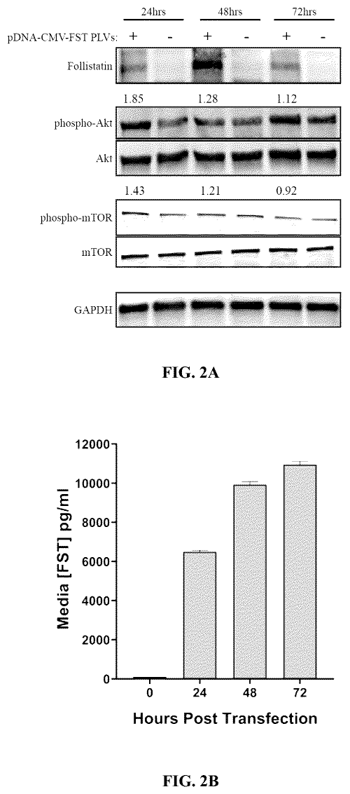

When pDNA-CMV-FST FAST-PLVs were incubated with C2C12 mouse myoblasts, robust FST expression was observed (FIG. 2A). FST expression correlated with increases in Akt and mTOR phosphorylation 24 and 48 hours after addition of pDNA-CMV-FST FAST-PLV (FIG. 2A; numbers above phosphorylated bands represent fold increase relative to pDNA-CMV-GFP treated C2C12 lysates).

FST protein in growth media was quantified by ELISA using a human follistatin ELISA kit (PeproTech, Cat. No. 900-K299) with slight modification to adapt it to the meso scale discovery (MSD) system. Capture FST antibody was coated on MSD standard binding plate at 1 μg/ml overnight at room temperature with shaking. The plate was washed three times with 0.05% Tween-20 in PBS followed by the addition of Blocker A (blocking buffer, MSD). After 1 hour of incubation, the plate was rewashed with PBS-T. Serially diluted Follistatin standard was prepared in Blocker A with 10% mouse serum. Mouse serum samples were prepared in Blocker A with a 1:10 dilution. The serum samples and follistatin standards were incubated overnight at 4° C. with shaking. The plate was rewashed with PBS-T and biotinylated follistatin detection antibody was added at a concentration of 1 μg/ml for 2 hours at room temperature with shaking. The plate was washed three times with PBS-T followed by the addition of 1 μg/mL sulfo-tag streptavidin for 1 hour at room temperature. The plate was washed with PBS-T three times, then Read buffer (Meso Scale Discovery) was added to the plate. Analysis was done with a MESO QuickPlex SQ 120.

The amount of FST in the growth media increased in a time-dependent manner, with levels reaching approximately 10,000 μg/mL 72 hours after addition of pDNA-CMV-FST FAST-PLV (FIG. 2B).

### E. Example 3: Systemic Administration of Expression Constructs Enhances Skeletal Muscle Size and Strength In Vivo

This example demonstrates that compositions and methods disclosed herein can elicit expression of FST and increased skeletal muscle size, strength, and cross-sectional area upon systemic administration.

The ability of systemically administered pDNA FAST PLVs using two different promoters to drive FST expression in vivo was examined. This example utilizes a mouse model for muscle wasting disorders, showing increased hindlimb muscle size and grip strength with follistatin gene therapy delivered via PLVs. PLVs containing expression constructs were administered to C57BL/6 mice at 10 mg/kg (of pDNA) via a single intravenous injection of 200 μL into the lateral tail vein.

As FST is primarily produced in the liver, expression when driven by the liver promoter transthyretin (TTR) was evaluated, and compared to the ubiquitous CMV promoter. An initial experiment compared serum expression of FST over time after administration of PLVs containing expression constructs with FST expression driven by: (i) a transthyretin (TTR) promoter, as shown in EXAMPLE 1; (ii) a CMV promoter; (iii) an ApoE promoter; or (iv) a SerpEnh promoter. Blood was collected via the lateral tail vein or cardiac puncture at indicated time points into serum collection tubes, and serum FST quantified via ELISA as outlined in EXAMPLE 2.

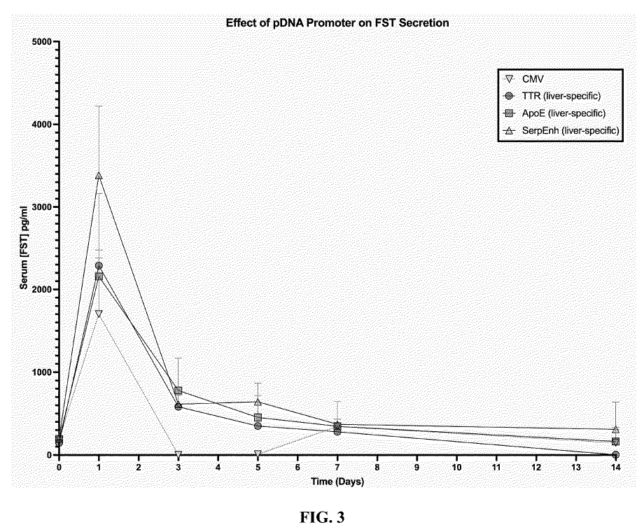

Intravenous administration of 10 mg/kg of the expression constructs resulted in a significant spike in serum FST concentration one day after injection, with reduced levels detected (e.g., near baseline) 3-7 days following administration (FIG. 3). On day one post-administration, the highest FST expression was observed for the SerpEnh construct. Animals receiving pDNA-CMV-FST PLVs reached a one-day peak serum FST concentration of approximately 1700 μg/mL, while animals receiving pDNA-TTR-FST FAST PLVs had a peak FST serum concentration of approximately 2300 μg/mL (FIG. 3). On days 3-14 post-administration, the highest FST expression was observed for the ApoE or SerpEnh constructs.

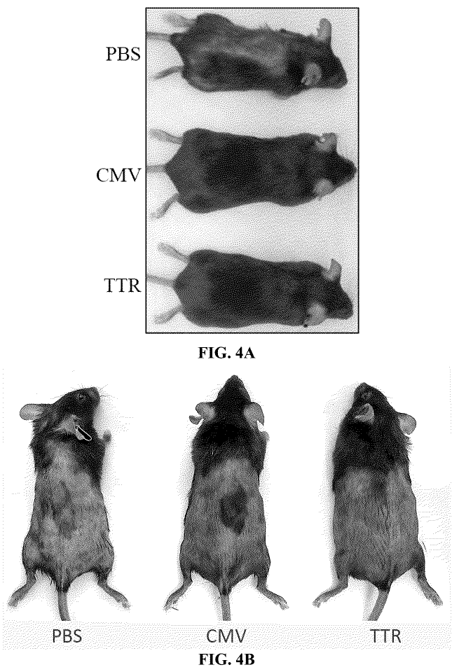

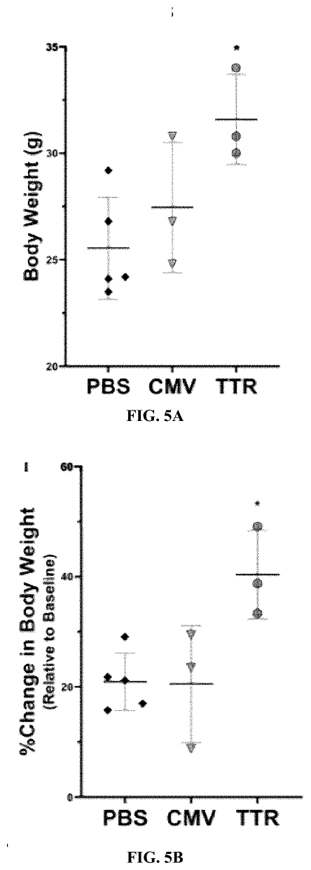

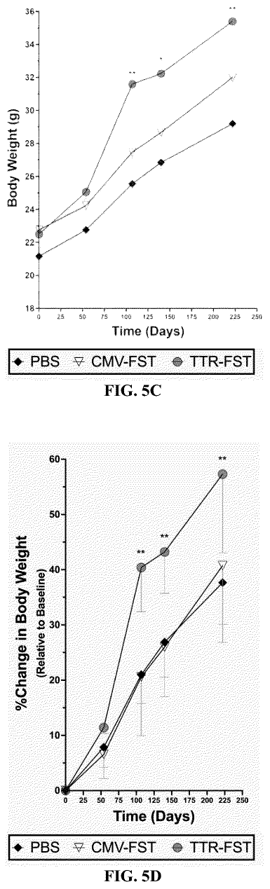

Additional studies evaluated longer term effects of the pDNA-TTR-FST and pDNA-CMV-FST constructs on skeletal muscle. After fifteen weeks, systemic administration of either gene therapy resulted in animals displaying a fuller frame and musculature, while PBS injected control animals appeared thinner with protruding pelvic and vertebral bones (FIG. 4A and FIG. 4B). Mice receiving pDNA-TTR-FST had significantly higher body weight, and percentage increases in body weight compared to PBS control mice, both at 15 weeks post-administration (FIG. 5A, FIG. 5B), and at other time points (FIG. 5C, FIG. 5D). This observed increase in body weight was consistent with significant increases in grip strength in these animals.

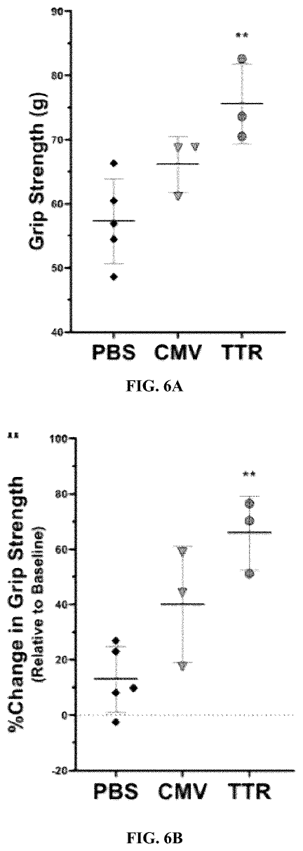

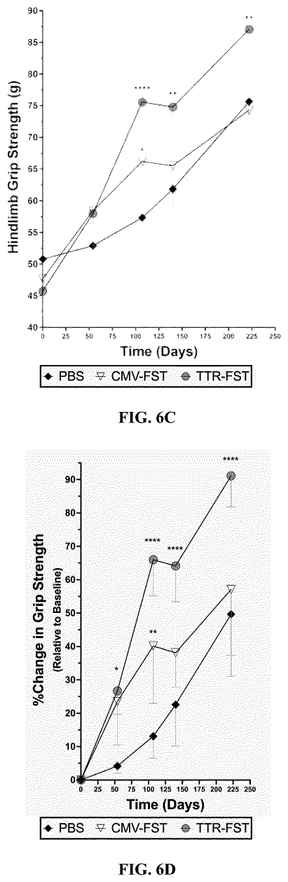

Hindlimb grip strength was measured in quintuplicate using a T-bar attachment on the BIOSEB grip strength meter. Mice receiving pDNA-TTR-FST exhibited significantly greater increases in grip strength and percentage increases in grip strength, both at 15 weeks post-administration (FIG. 6A, FIG. 6B), and at other time points (FIG. 6C, FIG. 6D).

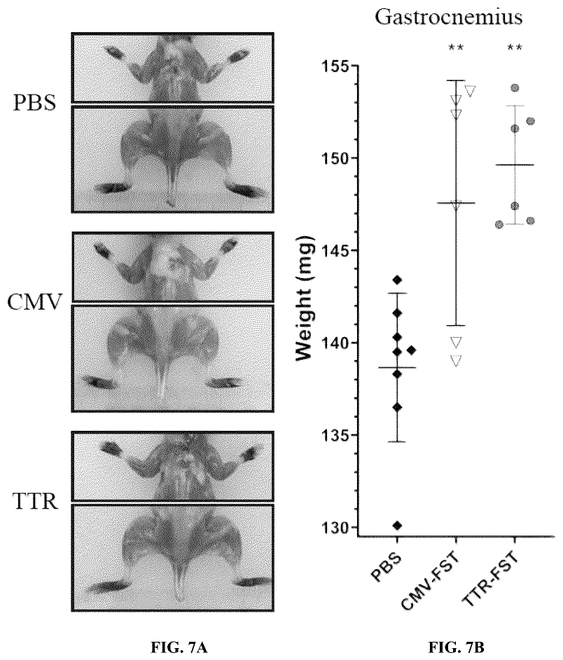

At 34 weeks post-injection, gross dissection revealed increased muscle size in mice receiving either FST construct, particularly in the hind limb (FIG. 7A). Gastrocnemius (GAS) muscles isolated from FST treated mice weighed significantly more than those isolated from PBS injected mice (FIG. 7B).

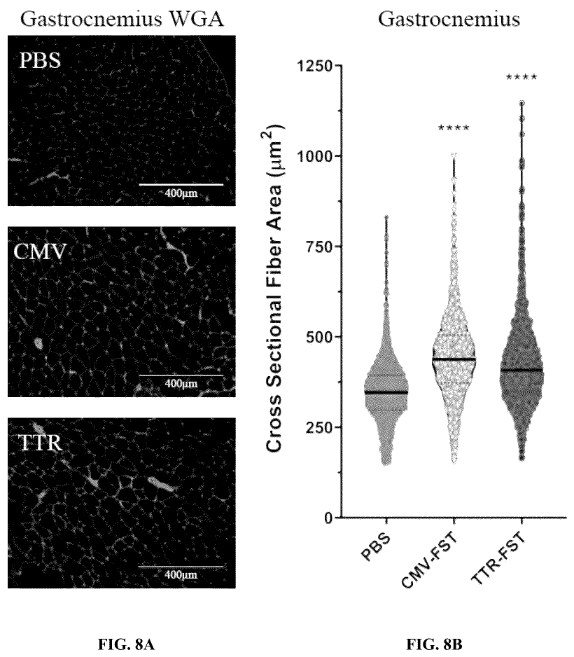

Wheat germ agglutinin (WGA) staining of gastrocnemius muscle was conducted to visualize and quantify muscular fiber cross-sectional area. Gastrocnemius utilized for determining muscle fiber area were flash-frozen in O.C.T. Compound and sectioned using a cryostat. 10 μm sections were warmed to room temperature and fixed with 3.7% formaldehyde for 15 minutes. Sections were washed three times with PBS. Sections were covered with 5 μg/ml wheat germ agglutinin Alexa Fluor-488 for 10 minutes at room temperature. Sections were then washed three times with PBS and mounted using ProLong Gold antifade reagent. Sections were visualized using an EVOS FL inverted microscope and 7-15 images were taken per section. Cross sectional muscle fiber area was determined using MyoVision software. Wheat germ agglutinin (WGA) staining of gastrocnemius muscle indicated that systemic FST administration resulted in significant increases in cross-sectional muscle fiber area (FIG. 8A and FIG. 8B).

These data demonstrate that compositions and methods disclosed herein can elicit expression of FST and increased skeletal muscle size, strength, and cross-sectional area upon systemic administration. Surprisingly, an expression construct utilizing a liver-specific promoter exhibited therapeutic effects with superior magnitude and duration compared to a strong ubiquitous/constitutive promoter (e.g., see FIGS. 5A-D and FIGS. 6A-D showing greater increases in body weight and grip strength). The therapeutic activity of the expression construct with the liver-specific promoter was remarkably durable following a single intravenous injection of a non-integrating expression construct (e.g., see FIG. 5D and FIG. 6D, with effects on size and grip strength persisting at least past day 200 post-administration).

These data also demonstrate that FAST-PLVs administered systemically can effectively deliver a therapeutic pDNA payload to generate body-wide effects.

### F. Example 4: Local or Systemic Administration of Expression Constructs Enhances Skeletal Muscle Size and Strength In Vivo

This example demonstrates that compositions and methods disclosed herein can elicit expression of FST and increased skeletal muscle size, strength, and cross-sectional area upon local or systemic administration.

The relative impact of local and systemic administration of the FAST-PLV FST gene therapy was examined. C57BL/6 mice were administered pDNA-CMV-FST FAST-PLV locally by single intramuscular injection of 5 mg/kg in the left hind limb gastrocnemius (GAS) muscle or systemically at 10 mg/kg. For local administration, mice received a single 50 μL intramuscular injection.

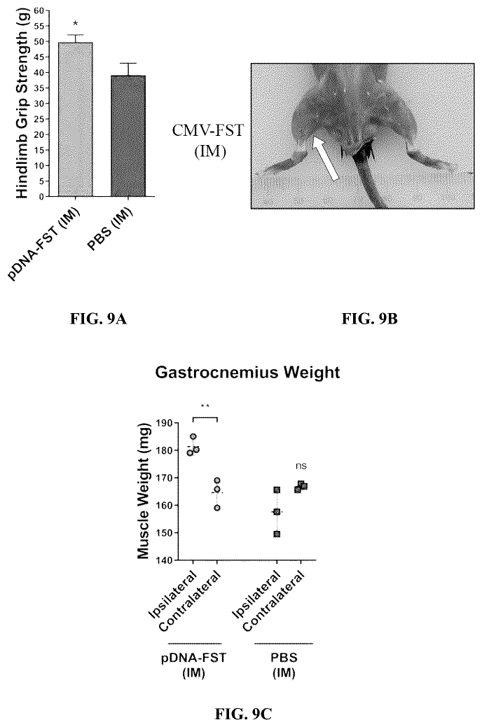

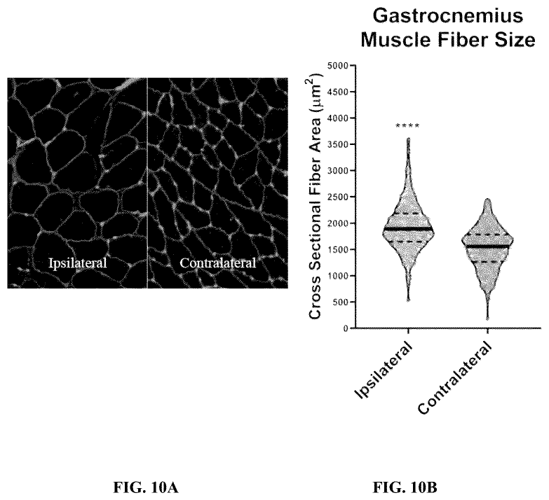

Relative to mice administered a volume matched PBS control, intramuscular administration of the expression construct resulted in a significant increase in hind limb grip strength (FIG. 9A) at 46 weeks post-injection. Gross dissection indicated that localized FST administration and expression resulted in a significant increase in the injected (ipsilateral) gastrocnemius size and weight relative to the non-injected (contralateral) gastrocnemius (FIG. 9B, FIG. 9C). Intramuscular injection also resulted in a significant increase in the cross-sectional muscle fiber area of the ipsilateral gastrocnemius relative to the contralateral gastrocnemius as shown by WGA staining (FIG. 10A, FIG. 10B).

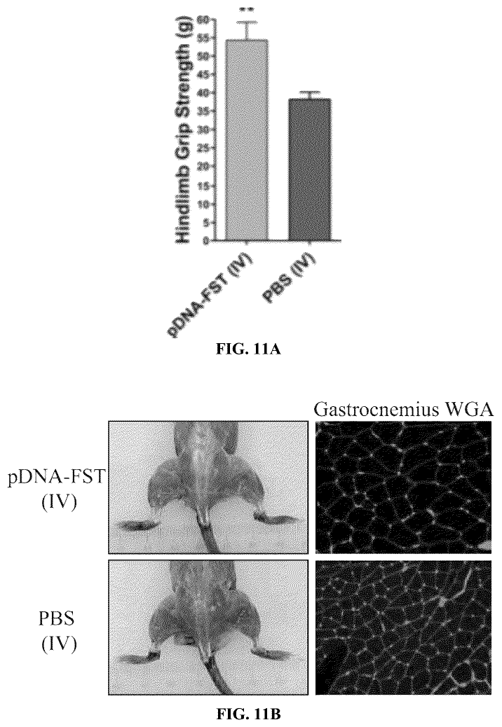

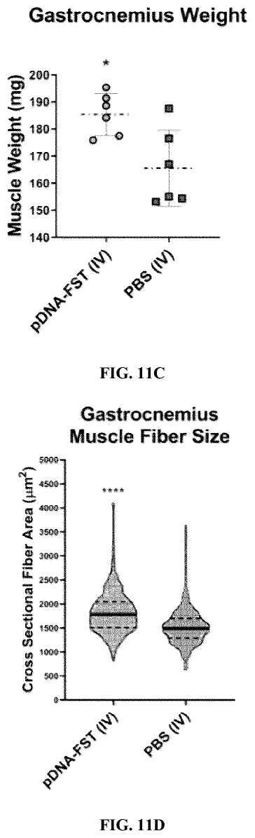

After systemic intravenous administration of 10 mg/kg pDNA-CMV-FST, a significant increase in hind limb grip strength was observed (FIG. 11A). Gross dissection revealed a significant increase in hindlimb muscle size and GAS weight (FIG. 11B, FIG. 11C). Relative to control animals, WGA staining of gastrocnemius demonstrated a significant increase in cross-sectional muscle fiber area (FIG. 11B, FIG. 11D).

These data demonstrate that local or systemic administration of an expression construct can result in FST expression and increased size, weight, strength, and cross-sectional area of injected muscle, and that the effects can persist long-term.

These data also demonstrate that FAST-PLVs administered locally can effectively deliver a therapeutic pDNA payload to generate localized effects.

1-74. (canceled)

75. A method of promoting retention of muscle mass or muscle strength or increasing muscle mass or muscle strength in a subject, the method comprising administering to the subject an effective amount of a non-viral delivery vector that comprises an expression construct comprising an expression regulatory region operatively linked to a transgene that encodes follistatin, thereby promoting the retention of muscle mass or muscle strength or increasing the muscle mass or muscle strength in the subject.

76. The method of claim 75, wherein the subject retains muscle strength at least 10% more than a control subject that is not administered the expression construct.

77. The method of claim 75, wherein the subject retains mass of a muscle at least 10% better than a control subject that is not administered the expression construct.

78. The method of claim 75, wherein the subject retains total muscle mass at least 10% better than a control subject that is not administered the expression construct.

79. The method of claim 75, wherein the non-viral delivery vector comprising the expression construct is administered to the subject prior to a surgery, a period of immobilization, or a period of reduced mobility.

80. The method of claim 75, wherein the subject has reduced mobility.

81. The method of claim 75, wherein the method treats sarcopenia in the subject.

82. The method of claim 75, wherein the method increases muscular strength, reduces loss of muscular strength, or promotes retention of muscular strength in the subject.

83. The method of claim 75, wherein the method treats age-related muscle loss, alpha-glucosidase deficiency, Becker muscular dystrophy, Duchenne muscular dystrophy (DMD), facioscapulohumeral muscular dystrophy, inclusion body myositis (IBM), inflammatory myopathy, muscular atrophy, muscular dystrophy, myopathy, myotonic dystrophy, or sporadic inclusion body or myositis (sIBM) in the subject.

84. The method of claim 75, wherein the administering is systemic.

85. The method of claim 75, wherein the administering is local.

86. The method of claim 75, wherein the subject is human.

87. The method of claim 75, wherein the expression construct is a DNA plasmid, nanoplasmid, or minicircle.

88. The method of claim 75, wherein the non-viral delivery vector is a proteo-lipid vehicle (PLV), a lipid nanoparticle (LNP), or a liposome.

89. The method of claim 88, wherein the non-viral delivery vector comprises a fusogenic protein.

90. The method of claim 75, wherein the non-viral delivery vector is the PLV and comprises an ionizable lipid and a fusion associated small transmembrane (FAST) protein.

91. The method of claim 75, wherein the follistatin comprises: (a) an amino acid sequence with at least 90% sequence identity to any one of SEQ ID NOs: 23-29, or (b) an amino acid sequence with at least 90% sequence identity to SEQ ID NO: 9 or SEQ ID NO: 18.

92. The method of claim 75, wherein the expression regulatory region comprises a tissue-specific or cell type-specific promoter.

93. The method of claim 75, wherein the expression regulatory region induces preferential expression of the transgene in liver tissue.

94. The method of claim 75, wherein the expression regulatory region comprises:
(a) from 5′ to 3′, a CRM8 element and an engineered transthyretin enhancer (TTRe);
(b) from 5′ to 3′, the TTRe and a human transthyretin promoter (hTTRm);
(c) from 5′ to 3′, the CRM8 element, the TTRe, and the hTTRm; or
(d) a nucleotide sequence with at least 90% sequence identity to SEQ ID NO: 11.

95. An expression construct comprising an expression regulatory region operatively linked to a transgene that encodes follistatin, wherein the expression regulatory region comprises, from 5′ to 3′: (i) a CRM8 element and an engineered transthyretin enhancer (TTRe); or (ii) the TTRe and a human transthyretin promoter (hTTRm).

96. An expression construct comprising an expression regulatory region operatively linked to a transgene that encodes follistatin, wherein the expression regulatory region comprises a human transthyretin promoter (hTTRm) nucleotide sequence with at least 90% sequence identity to SEQ ID NO: 4.
